(19) United States
(12) Patent Application Publication
(10) Pub. No.: US 2024/0124550 A1
(43) Pub. Date: Apr. 18, 2024

(54) RECOMBINANT IMMUNE CELLS, METHODS OF MAKING, AND METHODS OF USE

(71) Applicant: Regents of the University of Minnesota, Minneapolis, MN (US)

(72) Inventors: Jianming Wu, Plymouth, MN (US); Bruce Kenneth Walcheck, Lino Lakes, MN (US); Robert Harrison Hullsiek, Minneapolis, MN (US); Yungfang Li, Minneapolis, MN (US); Hemant Kumar Mishra, Minneapolis, MN (US)

(73) Assignee: REGENTS OF THE UNIVERSITY OF MINNESOTA, Minneapolis, MN (US)

(21) Appl. No.: 18/474,781

(22) Filed: Sep. 26, 2023

Related U.S. Application Data
(62) Division of application No. 16/758,142, filed on Apr. 22, 2020, now abandoned.
(60) Provisional application No. 62/577,425, filed on Oct. 26, 2017.

(51) Int. Cl. C07K 14/735; C07K 16/30; C12N 5/0783

(52) U.S. Cl. CPC C07K 14/70535; C07K 16/30; C12N 5/0646; C07K 2317/24; C07K 2319/74

(57) ABSTRACT

A recombinant immune cell expresses a heterologous IgG Fc receptor. In some embodiments, the heterologous IgG Fc receptor can be a chimeric IgG Fc receptor. Generally, the chimeric IgG Fc receptor includes an extracellular domain, a transmembrane domain, and an intracellular domain. The extracellular domain generally includes a sufficient portion of CD64 to bind to an IgG Fc region. The intracellular domain of the chimeric IgG Fc receptor includes a sufficient portion of an Fc receptor allowing immunoreceptor tyrosine-based activation motif (ITAM) to initiate cell signaling when an IgG Fc region binds to the extracellular domain.

### CROSS-REFERENCE TO RELATED APPLICATION

This application is a divisional of U.S. application Ser. No. 16/758,142, filed Apr. 22, 2020, which is a National Stage Application under 35 U.S.C. § 371 that claims the benefit of Application Serial No. PCT/US2018/057689, filed Oct. 26, 2018, which also claims the benefit of U.S. Provisional Application Ser. No. 62/577,425, filed Oct. 26, 2017. The disclosures of the prior applications are considered part of (and are incorporated by reference in) the disclosure of this application.

### GOVERNMENT FUNDING

This invention was made with government support under CA203348 awarded by National Institutes of Health. The government has certain rights in the invention.

### SEQUENCE LISTING

This application contains a Sequence Listing that has been submitted electronically as an XML file named 09531-0458002_SL_ST26.xml. The XML file, created on Sep. 25, 2023, is 40,708 bytes in size. The material in the XML file is hereby incorporated by reference in its entirety.

### SUMMARY

This disclosure describes, in one aspect, this disclosure describes an immune cell that expresses a heterologous IgG Fc receptor.

In some embodiments, the heterologous IgG Fc receptor can be a chimeric IgG Fc receptor. Generally, the chimeric IgG Fc receptor includes an extracellular domain, a transmembrane domain, and an intracellular domain. The extracellular domain generally includes a sufficient portion of CD64 to bind to an IgG Fc region. The intracellular domain of the chimeric IgG Fc receptor includes a sufficient portion of an Fc receptor immunoreceptor tyrosine-based activation motif (ITAM) to initiate cell signaling when an IgG Fc region binds to the extracellular domain.

In some of these embodiments, the intracellular domain includes at least a portion of the intracellular region of CD16A. In other embodiments, the intracellular domain can include at least a portion of the intracellular region of CD27, CD28, CD134 (OX40), CD137 (4-1BB), FcRγ, or CD3ζ.

In some embodiments, the chimeric IgG Fc receptor can include the CD16A extracellular cleavage site. In other embodiments, the extracellular domain of the chimeric IgG Fc receptor can lack the CD16A extracellular cleavage site.

In some embodiments, the heterologous IgG Fc receptor can include an IgG Fc receptor not natively expressed by the immune cell. In some of these embodiments, the immune cell may be a natural killer (NK) cell genetically modified to express CD64.

In another aspect, this disclosure describes a polynucleotide that encodes any embodiment of the heterologous IgG Fc receptors summarized above.

In another aspect, this disclosure describes an immune cell that is genetically modified to include the polynucleotide that encodes an embodiment of the heterologous IgG Fc receptors summarized above.

In another aspect, this disclosure describes a method of killing a tumor cell. Generally, the method includes contacting the tumor cell with an antibody that specifically binds to the tumor cell and contacting the tumor cell with any embodiment of the recombinant immune cell summarized above under conditions effective for the recombinant immune cell to kill the tumor cell.

In another aspect, this disclosure describes a method of treating a subject having a tumor. Generally, the method includes administering to the subject an antibody that specifically binds to cells of the tumor and administering to the subject a composition that includes any embodiment of the recombinant immune cell summarized above under conditions effective for the recombinant immune cell to kill cells of the tumor.

In another aspect, this disclosure describes a composition that include a complex formed between a therapeutic antibody and any embodiment of the recombinant immune cell summarized above in which the heterologous IgG Fc receptor is bound to the Fc portion of the therapeutic antibody.

In another aspect, this disclosure describes a method of treating a subject having a tumor. Generally, the method includes administering to the subject any embodiment of the composition summarized immediately wherein the therapeutic antibody specifically binds to cells of the tumor.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. Antibody-dependent cell-mediated cytotoxicity (ADCC).

FIG. 2. Wildtype CD16A, wildtype CD64, and the CD64/16A chimeric construct. The scissors and dashed line shown for CD16A represent the extracellular proteolytic site for ectodomain shedding.

FIG. 3. NK92 cells expressing either wildtype CD16A or CD64/16A. (A) NK92-CD64/16A cells were stained with anti-CD16, anti-CD64, or control antibodies. (B) NK92-CD16A cells were stained with anti-CD16, anti-CD64, or control antibodies. (C) NK92-CD64/16A, NK92-CD16A, or NK92 parent cells were incubated with trastuzumab then anti-human IgG-APC second stage antibody or anti-human IgG-APC second stage antibody alone (control). All antibody staining levels were determined by flow cytometry.

FIG. 4. NK92 cells expressing CD64/CD16A induce higher levels of ADCC than wildtype CD16A. (A) A standard ADCC assay was performed in which trastuzumab (herceptin) was included in the assay. (B) A standard ADCC assay was performed in which the NK92-CD64/CD16A and NK92-CD16A cells were pre-incubated with trastuzumab and then the mAb was washed away prior to the effector cells being incubated with the SKOV-3 target cells.

FIG. 5. Flow cytometry data comparing phenotypic markers expressed by iNK-CD64/CD16A and iNK-pKT2 cells.

FIG. 6. Bar graph with data showing iNK-CD64/CD16A cells induce higher levels of ADCC than iNK-CD16A cells using SKOV-3 target cells.

FIG. 7. Bar graph with data showing iNK-CD64/CD16A, but not iNK-CD16A cells can be pre-loaded with a therapeutic mAb and mediate ADCC.

FIG. 8. Bar graph with data showing iNK-CD64/CD16A cells induce higher levels of ADCC than iNK-CD16A cells using MA148 target cells.

FIG. 9. Amino acid sequence of an exemplary CD64/CD16A chimeric IgG Fc receptor (SEQ ID NO:1).

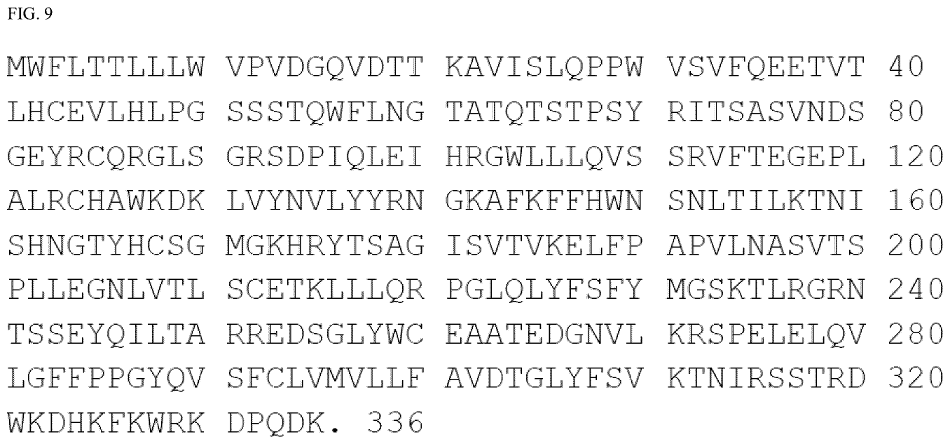

FIG. 10. Amino acid sequence of CD64 IgG Fc receptor (SEQ ID NO:2).

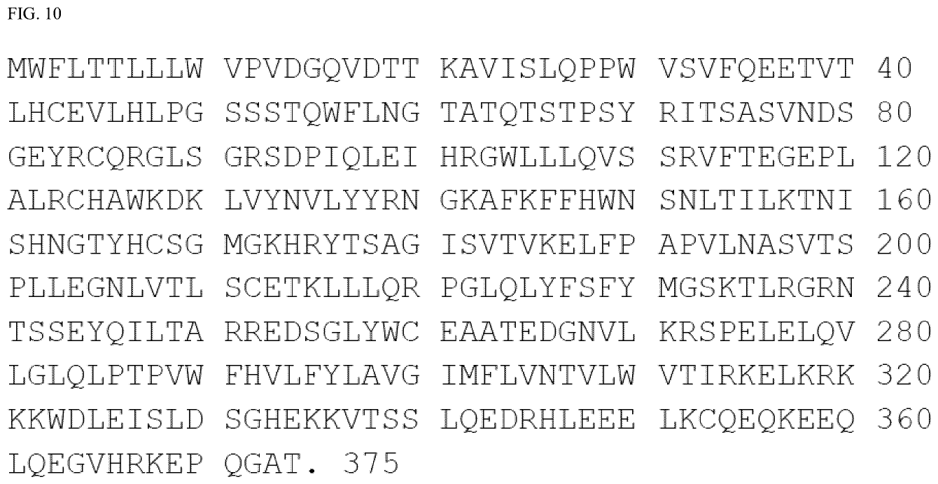

FIG. 11. Amino acid sequence of an exemplary CD16A-CD28-BB-ζ chain chimeric IgG Fc receptor (SEQ ID NO:3).

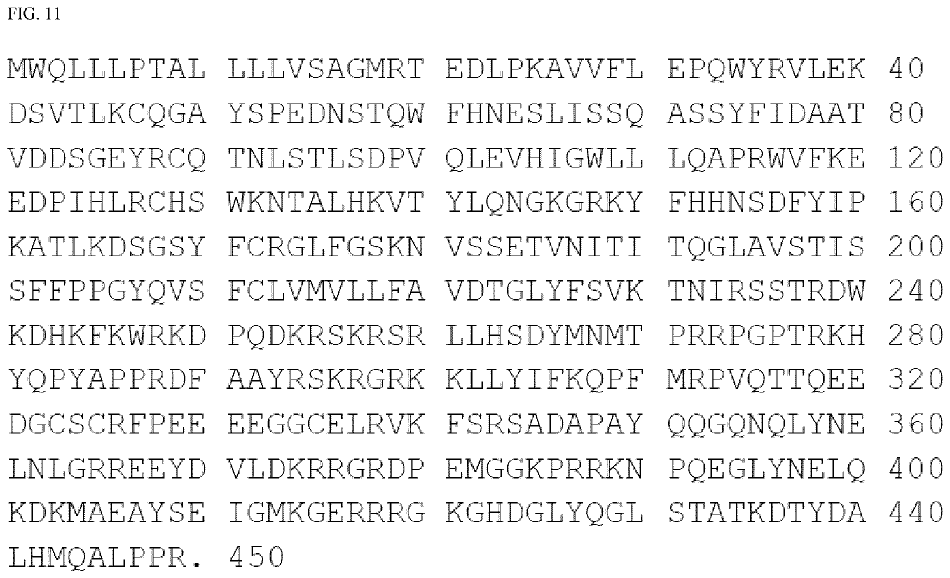

FIG. 12. Amino acid sequence of an exemplary CD16A-BB-ζ chain chimeric IgG Fc receptor (SEQ ID NO:4).

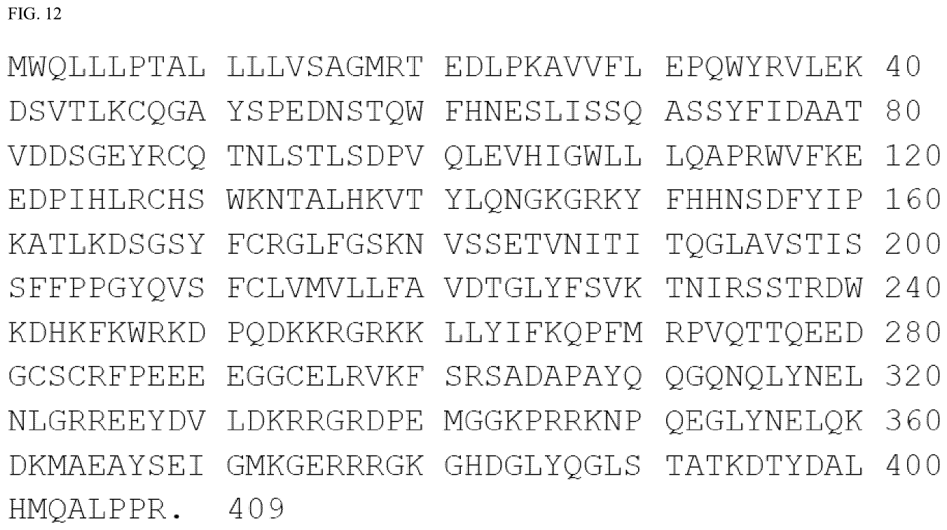

FIG. 13. Expression of CD64/16A by NK92 cells. (A) Schematic representation of the cell membrane forms of CD16A, CD64, and CD64/16A. CD16A undergoes ectodomain shedding by ADAM17 at a membrane proximal location, as indicated, which is not present in CD64 and CD64/16A. (B) NK92 parental cells, NK92-CD16A cells, and NK92-CD64/16A cells were stained with an anti-CD16, anti-CD64, or an isotype-matched negative control mAb and examined by flow cytometry. (C) NK92-CD16A and NK92-CD64/16A cells were incubated with SKOV-3 cells with or without trastuzumab (5 μg/ml) at 37° C. (E:T=1:1) for two hours. The NK92-CD16A and NK92-CD64/16A cells were then stained with an anti-CD16 mAb or an anti-CD64 mAb, respectively, and examined by flow cytometry. Nonspecific antibody labeling was determined using the appropriate isotype-negative control mAb. Data is representative of at least three independent experiments.

FIG. 14. CD64/16A promotes target cell conjugation, ADCC, and IFNγ production. (A) NK92-CD64/16A cells expressing eGFP and SKOV-3 cells labeled CellTrace Violet were mixed at an E:T ratio of 1:2 with or without trastuzumab (5 μg/ml), incubated at 37° C. for 60 minutes, fixed, and then analyzed by flow cytometry. Representative data from at least three independent experiments are shown. (B) NK92-CD64/16A cells were incubated with SKOV-3 cells (E:T=20:1) and trastuzumab (tras.) at the indicated concentrations (left panel), or with SKOV-3 cells at the indicated E:T ratios in the presence or absence of trastuzumab (5 μg/ml) (right panel) for two hours at 37° C. Data are represented as % specific release and the mean±SD of three independent experiments is shown. Statistical significance is indicated as *p<0.05, **p<0.01. (C) NK92-CD64/16A cells were incubated with SKOV-3 cells (E:T=20:1) in the presence or absence of trastuzumab (5 μg/ml) and the anti-CD64 mAb 10.1 (10 μg/ml), as indicated, for two hours at 37° C. Data are represented as % specific release and the mean±SD of three independent experiments is shown. Statistical significance is indicated as **p<0.01. (D) NK92-CD64/16A cells were incubated with SKOV-3 cells (E:T=1:1) with or without trastuzumab (5 μg/ml) for two hours at 37° C. Secreted IFNγ levels were quantified by ELISA. Data is shown as mean of two independent experiments.

FIG. 15. CD64/16A attaches to soluble tumor-targeting mAbs and IgG fusion proteins. (A) Relative expression levels of CD16A and CD64/16A on NK92 cells were determined by cell staining with anti-CD16 and anti-CD64 mAbs (black bars), respectively, or an isotype-matched negative control antibody (gray bars). The bar graph shows mean fluorescence intensity (MFI)±SD of three independent experiments. Representative flow cytometric data are shown in the histogram overlay. The dashed line histogram shows CD64 staining of NK92-CD64/16A cells, the orange-filled histogram shows CD16A staining of NK92-CD16A cells, and the green-filled histogram shows isotype control antibody staining of the NK92-CD16A cells. (B) NK92-CD16A and NK92-CD64/16A cells were incubated with or without trastuzumab (5 μg/ml) for two hours at 37° C., washed, stained with a fluorophore-conjugated anti-human secondary antibody, and analyzed by flow cytometry. Data is representative of at least three independent experiments. (C) NK92-CD64/16A cells were incubated with cetuximab or rituximab (5 μg/ml for each), washed, and then stained with a fluorophore-conjugated anti-human secondary antibody. Control represents cells stained with the anti-human secondary antibody only. NK92-CD64/16A cells were also incubated with L-selectin/Fc (5 μg/ml), washed, and then stained with a fluorophore-conjugated anti-L-selectin mAb. NK92 cells lack expression of endogenous L-selectin (data not shown). All staining was analyzed by flow cytometry. Data shown are representative of three independent experiments. (D) NK92-CD16A and NK92-CD64/16A cells were incubated in the presence or absence of trastuzumab (5 μg/ml), washed, and exposed to SKOV-3 cells at the indicated E:T cell ratios for two hours at 37° C. Data is shown as mean±SD of three independent experiments. Statistical significance is indicated as **p<0.01, ***p<0.001. bd=below detection, (i.e., <spontaneous release by negative control cells). (E) NK92-CD16A and NK92-CD64/16A cells were incubated with SKOV-3 cells (E:T=10:1) in the presence or absence of trastuzumab (5 μg/ml), as indicated, for two hours at 37° C. Data is shown as mean±SD of three independent experiments. Statistical significance is indicated as **p<0.01.

FIG. 16. Generation of iNK cells expressing CD64/CD16A. iPSCs were transduced to stably express CD64/16A, differentiated into NK cells, and then expanded using K562-mbIL21-41BBL feeder cells. iNK-CD64/16A cells and freshly isolated peripheral blood (PB) NK cells enriched from adult peripheral blood were stained for CD56, CD3 and various inhibitory and activating receptors, as indicated. CD64/16A expression was determined by staining the cells with an anti-CD64 mAb. Representative data from at least three independent experiments are shown.

FIG. 17. iNK-CD64/16A cells show enhanced ADCC compared to iNK-pKT2 control cells. (A) NK cells derived from empty vector (iNK-pKT2) or CD64/16A (iNK-CD64/16A) transduced iPSCs were stained for CD56, CD64, and CD16A, as indicated. (B) iNK-pKT2 and iNK-CD64/16A cells were incubated with SKOV-3 cells (E:T=10:1) in the presence or absence of trastuzumab (5 μg/ml), the function blocking anti-CD16 mAb 3G8 (5 μg/ml), and the function blocking anti-CD64 mAb 10.1 (5 μg/ml), as indicated, for two hours at 37° C. Data is shown as mean±SD of three independent experiments. Statistical significance is indicated as ***p<0.001; ****p<0.0001. (C) iNK-pKT2 and iNK-CD64/16A cells were incubated in the presence or absence of trastuzumab (5 μg/ml), washed, and exposed to SKOV-3 cells (E:T=10:1) for two hours at 37° C. Data is shown as mean±SD of three independent experiments. Statistical significance is indicated as ***p<0.001.

FIG. 18. Sequence alignment of canine CD16A (SEQ ID NO:5), canine CD64sp (SEQ ID NO:25), and human CD16A (SEQ ID NO:6).

FIG. 19. Sequence alignment of canine CD64 (SEQ ID NO:7) and human CD64 (SEQ ID NO:8).

FIG. 20. NK92 cells expressing wildtype human CD64 mediate ADCC. (A) NK92-CD64 cells were stained with an isotype-matched negative control mAb or the anti-CD64 mAb (clone 10.1) and examined by flow cytometry. (B) NK92-CD64 cells were incubated with SKOV-3 cells (at the indicated E:T ratios) in the presence or absence of trastuzumab (tras.) (5 μg/ml) for two hours at 37° C. Representative data from at least three independent experiments are shown.

### DETAILED DESCRIPTION OF ILLUSTRATIVE EMBODIMENTS

This disclosure describes recombinant immune cells, methods of making the recombinant immune cells, and methods of using the recombinant immune cells. Generally, the recombinant immune cells are genetically modified to include a heterologous IgG Fc receptor. In some cases, the heterologous IgG Fc receptor can be a chimeric receptor engineered include domains from two or more receptors. In other embodiments, the heterologous may be an IgG Fc receptor that is not natively expressed by the immune cell. Generally, the recombinant immune cells provide a sustained cytotoxic immune response against a target—for example, a tumor cell—that is targeted for killing by the immune cell because the target binds a therapeutic antibody that is recognized by the IgG Fc receptor.

A mechanism of cell-mediated immune defense involves the engagement of antibodies attached to target cells by Fc receptors expressed by leukocytes, which results in target cell killing. This process is referred to antibody-dependent cell-mediated cytotoxicity (ADCC). Therapeutic monoclonal antibodies (mAbs) have been generated against a variety of tumor antigens and tested in clinical trials for treating infectious diseases, chronic diseases, and cancers including, for example, AML, breast cancer, ovarian cancer, gastric cancer, neuroblastoma, and lymphoma. Many clinically successful mAbs use ADCC as a mechanism of action. A limitation of antibody therapy, however, is the development of resistance in patients and the non-responsiveness of some malignancies.

This disclosure describes an approach for augmenting Fc receptor interactions with therapeutic antibodies. The approach involves a chimeric receptor that includes a CD16A domain and a CD64 domain.

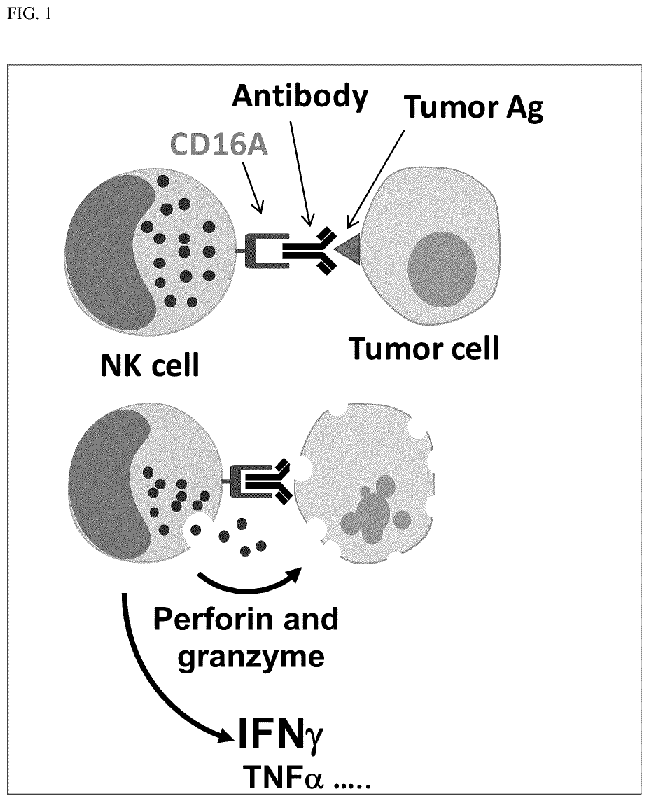

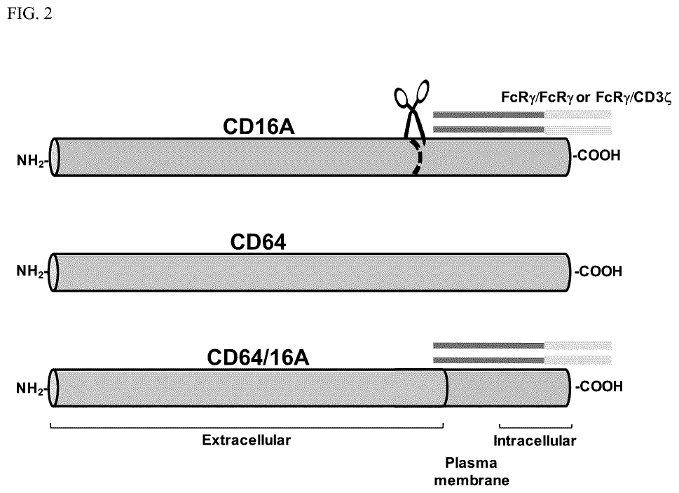

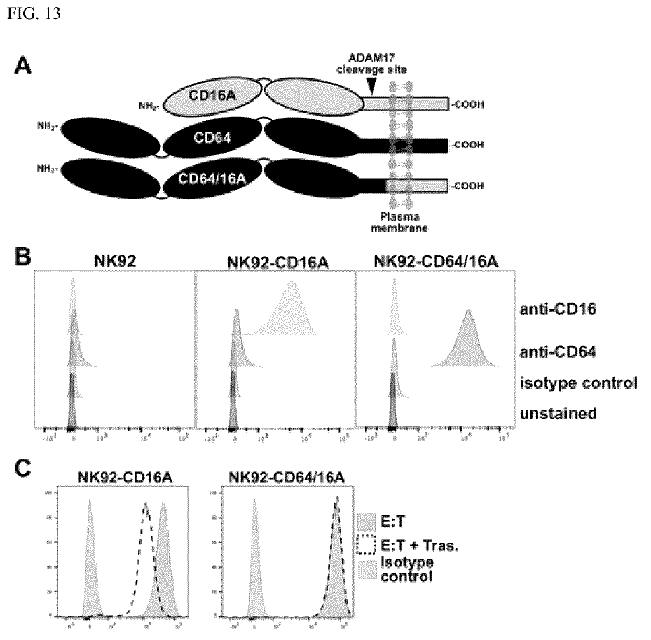

CD16A (FcγRIIIA) is an IgG Fc receptor expressed by human natural killer (NK) cells, a population of cytotoxic lymphocytes, and is their sole means of recognizing IgG bound to tumor cells or virus-infected cells. CD16A is a potent activating receptor that induces ADCC by NK cells (FIG. 1). The CD16A transmembrane region is responsible for the association with CD3ζ and/or FcR γ-chain (FcRγ) that contain immunoreceptor tyrosine-based activation motifs (FIG. 2; FIG. 13A), and the CD16A cytoplasmic domain interacts with intracellular molecules critical for receptor functions. CD16A is a low affinity FcγR with limited capacity to engage therapeutic mAb-coated target cells. CD16A also undergoes a rapid downregulation in expression upon cell activation that markedly reduces its cell surface density and avidity for IgG. CD16A downregulation occurs by a proteolytic event at an extracellular site proximal to the plasma membrane and is referred to as ectodomain shedding. The location of this cleavage site has been reported (Jing et al., 2015. PLoS One 10:e0121788) and is shown schematically in FIG. 2 and FIG. 13A.

CD64 (FcγRI) is another IgG Fc receptor, and is expressed by monocytes, macrophages, and activated neutrophils. CD64 is a high affinity IgG receptor. This receptor does not undergo ectodomain shedding upon cell activation and does not naturally transduce signals for ADCC in NK cells.

This disclosure describes a chimeric FcγR that includes a CD16A domain and a CD64 domain. The chimeric receptor includes the extracellular region of human CD64 and the cytoplasmic region of human CD16A, an exemplary embodiment of which is shown schematically in FIG. 2 and FIG. 13A as CD64/16A. In various embodiments, the chimeric CD64/CD16A receptor can include the CD64 transmembrane region or the CD16A transmembrane region. The CD64/16A construct has been engineered so that it lacks the CD16A extracellular cleavage site and thus is not susceptible to ectodomain shedding (FIG. 2; FIG. 13A), but includes at least a portion of the CD16A intracellular region that is involved in intracellular signaling.

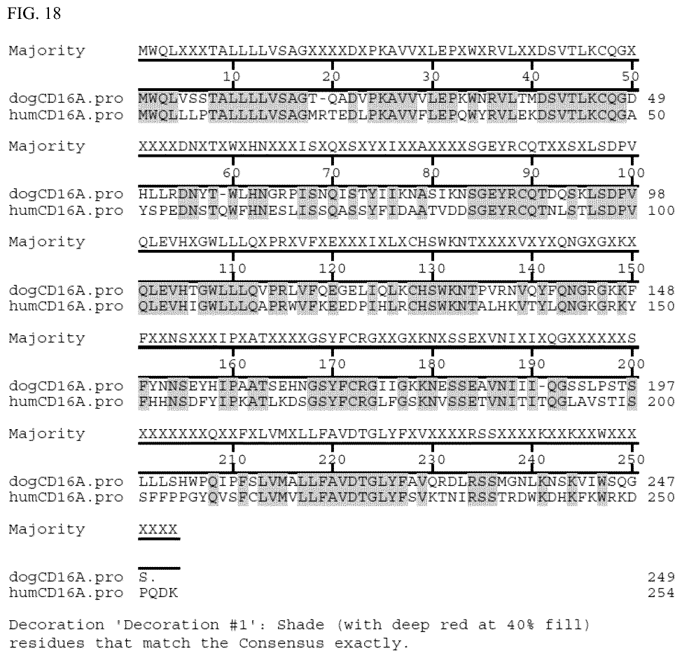

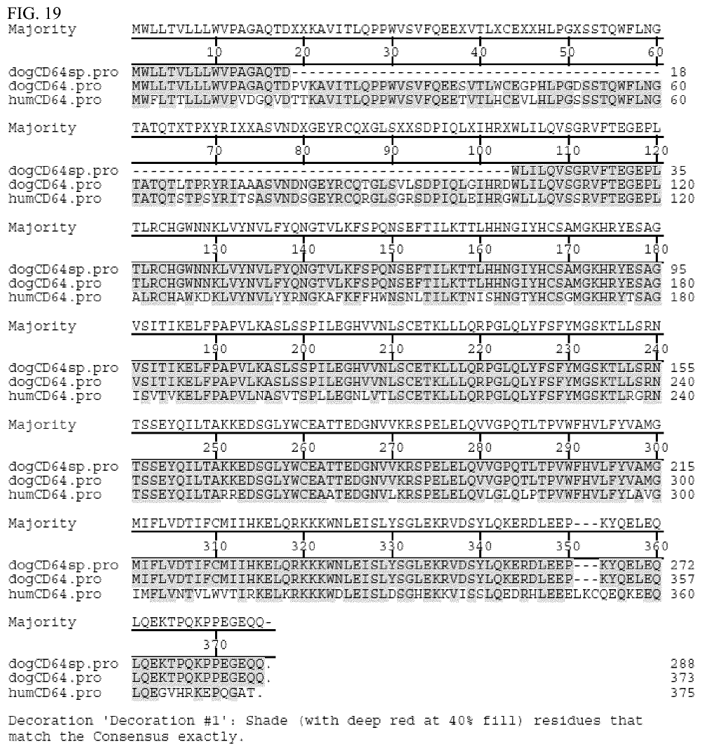

Also, while described herein in the context of an exemplary embodiment in which the CD64 domain and the CD16A contain amino acid sequences of human CD64 and human CD16A, respectively, the chimeric FcγR described herein can include an amino acid sequence that is, or is derived from, any suitable CD64 or CD16A natively expressed by any species. FIG. 18 and FIG. 19 provide amino acid sequence alignments of human and canine amino acid sequences for CD16A (FIG. 18) and CD64 (FIG. 19).

As used herein, the amino acid sequence of a domain is “derived from” a the amino acid sequence of a reference polypeptide if the amino acid sequence of the domain possesses a specified amount of sequence similarity and/or sequence identity compared to the amino acid sequence of the reference polypeptide. Sequence similarity of can be determined by aligning the residues of the two polypeptides (for example, the domain amino acid sequence and the amino acid sequence of the reference CD16A or CD64 polypeptide) to optimize the number of identical amino acids along the lengths of their sequences. Gaps in either or both sequences are permitted in making the alignment in order to optimize the number of identical amino acids, although the amino acids in each sequence must nonetheless remain in their proper order.

A pair-wise comparison analysis of amino acid sequences can be carried out using the BESTFIT algorithm in the GCG package (version 10.2, Madison WI). Alternatively, polypeptides may be compared using the Blastp program of the BLAST 2 search algorithm, as described by Tatiana et al., (FEMS Microbiol Lett, 174, 247-250 (1999)), and available on the National Center for Biotechnology Information (NCBI) website. The default values for all BLAST 2 search parameters may be used, including matrix=BLOSUM62; open gap penalty=11, extension gap penalty=1, gap x_dropoff=50, expect=10, wordsize=3, and filter on.

The amino acid sequence of a domain is “derived from” a the amino acid sequence of a reference polypeptide if the amino acid sequence of the domain possesses a specified degree of amino acid sequence “identity” or amino acid sequence “similarity.” Amino acid sequence identity refers to the presence of identical amino acids. Amino acid sequence similarity refers to the presence of not only identical amino acids but also the presence of conservative substitutions. A conservative substitution for an amino acid may be selected from other members of the class to which the substituted amino acid belongs. For example, it is well-known in the art of protein biochemistry that an amino acid belonging to a grouping of amino acids having a particular size or characteristic (such as charge, hydrophobicity and hydrophilicity) can be substituted for another amino acid without altering the activity of a protein, particularly in regions of the protein that are not directly associated with biological activity. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Conservative substitutions include, for example, Lys for Arg and vice versa to maintain a positive charge; Glu for Asp and vice versa to maintain a negative charge; Ser for Thr so that a free —OH is maintained; and Gln for Asn to maintain a free —NH₂. Likewise, biologically active analogs of a polypeptide containing deletions or additions of one or more contiguous or noncontiguous amino acids that do not eliminate a functional activity of the polypeptide are also contemplated.

The amino acid sequence of a CD16A domain or a CD64 domain is “derived from” a reference amino acid sequence if the domain amino acid sequence has at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence similarity to the reference amino acid sequence.

The amino acid sequence of a CD16A domain or a CD64 domain is “derived from” a reference amino acid sequence if the domain amino acid sequence has at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the reference amino acid sequence.

For the purpose of determining whether a domain amino acid sequence is “derived from” a specified reference amino acid sequence, exemplary suitable reference polypeptides include human CD16A (SEQ ID NO:6), canine CD16A (SEQ ID NO:5), human CD64 (SEQ ID NO:8), canine CD64 (SEQ ID NO:7), canine CD64sp (SEQ ID NO:25), or the corresponding domains of any of the constructs listed in Table 1.

Also, while described herein in the context of an exemplary embodiment, illustrated in FIG. 2 and FIG. 13A, the chimeric receptor may be designed to include a different fusion points between CD64 and CD16A that that shown in FIG. 2 and FIG. 13A, to include the CD16A cleavage region, modified functional motifs in regions of CD64 or CD16A, and/or adding an additional signaling domain, such as, for example, a signaling domain of CD27, CD28, CD134 (OX40), CD137 (4-1BB), FcRγ, or CD3ζ. The chimeric receptor may therefore be designed to increase the proliferation of NK cells or other effector cells, increase survival of NK cells or other effector cells, increase potency of NK cells or other effector cells, and/or decrease NK cell exhaustion in vivo.

In certain embodiments, the chimeric FcγR can be modified to include a cytoplasmic domain or a signaling domain that confers additional functionality to the chimeric receptor. For example, a chimeric FcγR can include a functional portion of CD28, which transduces signals involved in T-cell proliferation, survival, and cytokine production. As another example, a chimeric FcγR can include a functional portion of 4-1BB, which contributes to the clonal expansion, survival, and development of hematopoietic cells. As another example, a chimeric FcγR can include a functional portion of the CD3ζ cytoplasmic domain, which contains three immunoreceptor tyrosine-based activation motifs (ITAMs) that trigger intracellular signal-transduction pathways for ADCC, cytokine production, and cell proliferation and survival. As another example, a chimeric FcγR can include a functional portion of the FcRγ cytoplasmic domain, which contains an ITAM that preferentially recruits Syk kinase to mediate intracellular signals for ADCC, cytokine production, and cell proliferation and survival. As another example, a chimeric FcγR can include a functional portion of the DAP10 cytoplasmic domain, which contains a YxxM motif that specifically activates phosphatidylinositol 3-kinase-dependent signaling pathways for cytotoxicity, cell survival, and proliferation of NK and T cells. As another example, a chimeric FcγR can include a functional portion of the DAP12 cytoplasmic domain, which contains an ITAM that triggers signals for cytotoxicity, survival, and proliferation of NK cells and T cells. As another example, a chimeric FcγR can include a functional portion of the NKG2D (or CD314) transmembrane domain, which specifically associates with DAP10 to mediate signaling pathways for cytotoxicity, cell survival, and proliferation of NK and T cells. As another example, a chimeric FcγR can include a functional portion of the 2B4 (NKR2B4 or CD244) cytoplasmic domain, which transduces signals of cytolytic granule polarization involved in enhanced cytotoxicity of NK cells. As another example, a chimeric FcγR can include a functional portion of the high affinity IgE receptor FcεR transmembrane and cytoplasmic domains, which are constitutively associated with its β-subunit and FcR γ-chain (FcRγ) to mediate the most potent degranulation signals in myeloid cells that initiates the allergic responses, which could be exploited for cancer therapy with the recombinant high affinity IgG Fc receptors.

Exemplary constructs that include exemplary cytoplasmic domain and/or signaling domain modifications are listed in Table 1.

TABLE 1

| Code | Domains (EC/TM/CY/SDs)* | Comments | SEQ ID NO: |
|---|---|---|---|
| rCD64#1 | 64/64/64/— | | 9 |
| rCD64#2 | 64/64/mut64/— | CD64 cytoplasmic domain | 10 |
| | | mutation | |
| rCD64#3 | 64/16A/16A/— | | 11 |
| rCD64#4 | 64/16A/16A/28-BB-CD3ç | | 12 |
| rCD64#5 | 64/16A/16A/28-BB-FcRγ | | 13 |
| rCD64#6 | 64/16A/16A/28-BB-Dap10 | | 14 |
| rCD64#7 | 64/16A/16A/28-BB-Dap12 | | 15 |
| rCD64#8 | 64/28/28/BB-CD3ç | | 16 |
| rCD64#9 | 64/FcεR/FcεR/— | | 17 |
| rCD64#10 | 64/16A/mut16A/— | Mutation on PKC | 18 |
| | | phosphorylation site | |
| rCD64#11 | 64/G2D/2B4/CD3ç | | 19 |
| rCD64#12 | 64/16A/16A/2B4-CD3ç | | 20 |
| rCD64#13 | 64/16A/16A/2B4-FcRγ | | 21 |
| rCD64#14 | 64/16A/16A/2B4-Dap10 | | 22 |
| rCD64#15 | 64/16A/16A/2B4-Dap12 | | 23 |
| rCD64#16 | 64/64/64/2B4-CD3ç | | 24 |

*EC: Extracellular domain;
TM: Transmembrane domain;
CY: Cytoplasmic domain;
SD: Signaling domain.
64: CD64, High affinity IgG Fc receptor FcγRI;
mutCD64: High affinity IgG Fc receptor FcγR1 with cytoplasmic mutation that results in higher levels of cytokine production and degranulation;
16A: C16A, Low affinity IgG Fc receptor FcγRIIIA;
—: no signaling domain;
28: CD28, a co-stimulatory receptor for cell proliferation and activation;
BB: 4.1BB or CD137;
CD3ç: CD3 ç-chain or CD247;
FcRγ: FcR γ-chain;
D10: DAP10 signaling adaptor;
D12: DAP12 signaling adaptor;
FceR: High affinity IgE Fc receptor;
16-PKC⁻: CD16A cytoplasmic domain with mutations on PKC phosphorylation site to disrupt cytokine productions mediated by CD16A;
G2D: NKG2D or CD314;
2B4: NKR2B4 or CD244.

The CD64/16A chimeric receptor can be encoded by a cDNA that can be transcribed and translated from an expression vector introduced into a host cell to produce a recombinant cell. The host cell can include a suitable leukocyte-like cell or a primary leukocyte. Suitable leukocyte-like cells include, but are not limited to, a hematopoietic cell line or an induced pluripotent stem cell. Suitable primary leukocytes include, but are not limited to, NK cells, monocytes, macrophages, neutrophils, or T lymphocytes. The expression of CD64/16A by genetically-engineered leukocytes can increase the recombinant cell's effector function in killing target cells—e.g., tumor cells and virus-infected cells—in the presence of natural and therapeutic antibodies compared to the unmodified host cell. Because the CD64/16A chimeric receptor binds to IgG with high affinity, it may also be feasible to attach therapeutic mAbs to effector cells expressing the construct prior to their administration into patients. Hence, CD64/16A with an attached therapeutic mAb would provide the effector cells with a targeting element to direct them to cancer locations.

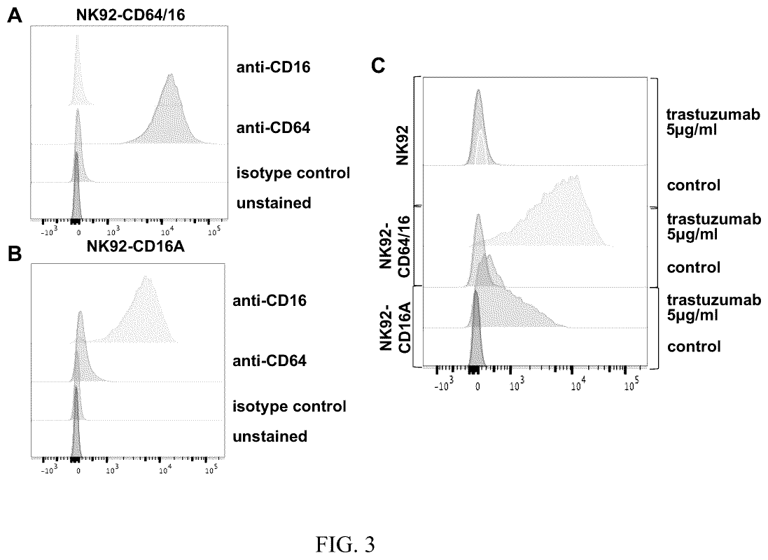

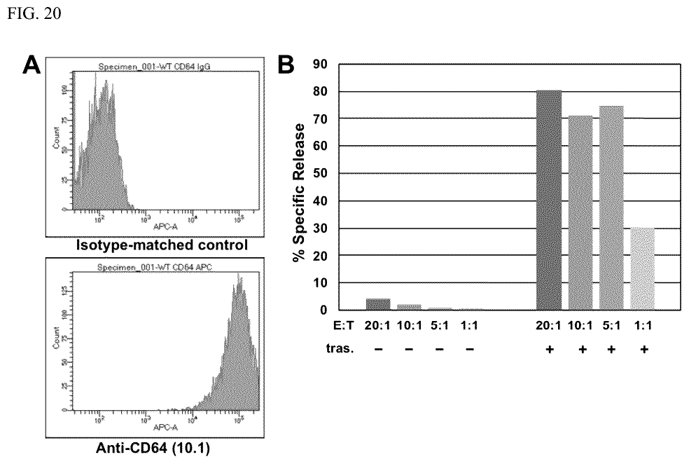

NK92 cells are a human NK cell line that lacks expression of endogenous CD16A. NK cells were generated that express in a stable manner wildtype CD16A, wildtype CD64, or the exemplary chimeric receptor CD64/16A. NK92 cells expressing CD64/16A could be stained with an anti-CD64 mAb, but not with an anti-CD16 mAb (FIG. 3A). NK92-CD16A cells could be stained with an anti-CD16 mAb, but not with an anti-CD64 mAb (FIG. 3B). NK92 cells expressing CD64 could be stained with an anti-CD64 mAb, but not by and isotype-matched negative control mAb (FIG. 20A).

FIG. 3C shows the ability of NK92 cells expressing CD64/16A or CD16A to bind trastuzumab, a therapeutic mAb specific to HER2/EGFR2 overexpressed by certain malignancies. Non-transduced NK92 cells, NK92-CD64/16A cells, and NK92-CD16A cells were incubated with trastuzumab (5 μg/ml) for two hours at room temperature, washed to remove unbound antibody, incubated with an anti-human IgG second stage antibody conjugated to a fluorophore, and then examined by flow cytometry. NK92-hCD64/16A cells bound much higher levels of trastuzumab than did NK92-CD16A cells and NK92 cells.

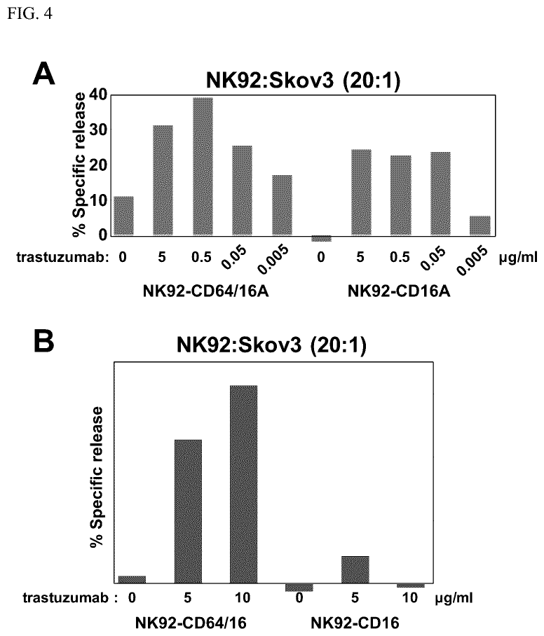

FIG. 4 presents data that shows the exemplary chimeric receptor CD64/16A conferred NK92 cells with an ADCC effector function. NK92 cells expressing CD64/16A or wildtype CD16A were incubated with the human ovarian cancer cell line SKOV-3 (20:1 ratio) in the presence or absence of trastuzumab at various concentrations (0.005 μg/ml, 0.05 μg/ml. 0.5 μg/ml, or 5 μg/ml). NK92 cells expressing either CD64/16A or wildtype CD16A demonstrated SKOV-3 cytotoxicity in the presence of trastuzumab. NK92 cells expressing CD64/16A had higher levels of target cell killing than did NK92-CD16A cells at all trastuzumab concentrations examined (FIG. 4A). NK92 cells expressing either CD64 also demonstrated SKOV-3 cytotoxicity in the presence of trastuzumab (FIG. 20B). In addition, NK92-CD64/16A and NK92-CD16A cells were pretreated with trastuzumab at 5 μg/ml or 10 μg/ml for two hours, washed to remove unbound antibody, and then incubated with SKOV-3 cells. In this assay, NK92-CD64/16A cells demonstrated a marked enhancement in target cell killing when compared to NK92-CD16A cells (FIG. 4B).

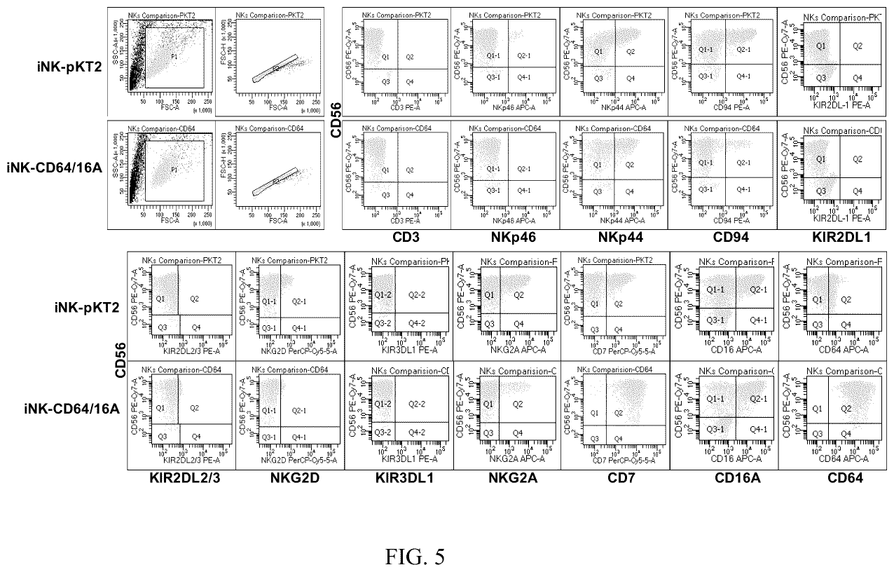

CD64/16A also was expressed in iPSCs and these cells were then differentiated into NK cells (referred to here as iNK cells). As shown in FIG. 5, iNK cells transduced with either CD64/16A or the empty vector (pKT2) as a control were compared for their expression of several NK cell markers. iNK-CD64/16A and iNK-pKT2 cells are CD56⁺ and CD3⁻, indicating that they are indeed NK cells. They also expressed similar levels of various NK cell markers. iNK-CD64/16A and iNK-pKT2 cells were found to express similar levels of CD16A, whereas only iNK-CD64/16A were stained by an anti-CD64 mAb (FIG. 5).

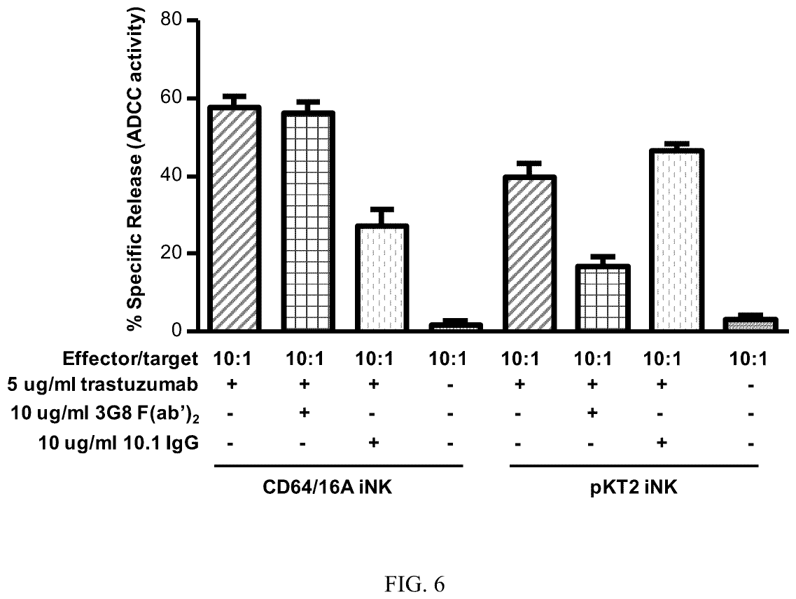

iNK-CD64/16A and iNK-pKT2 cells were evaluated for ADCC, as described above for the NK92 cells. iNK-CD64/16A and iNK-pKT2 cells were incubated with SKOV-3 cells at 10:1 ratio in the presence or absence of trastuzumab (5 μg/ml). iNK-CD64/16A cells demonstrated increased SKOV-3 cytotoxicity in the presence of trastuzumab and higher levels of ADCC when compared to iNK-pKT2 cells (FIG. 6).

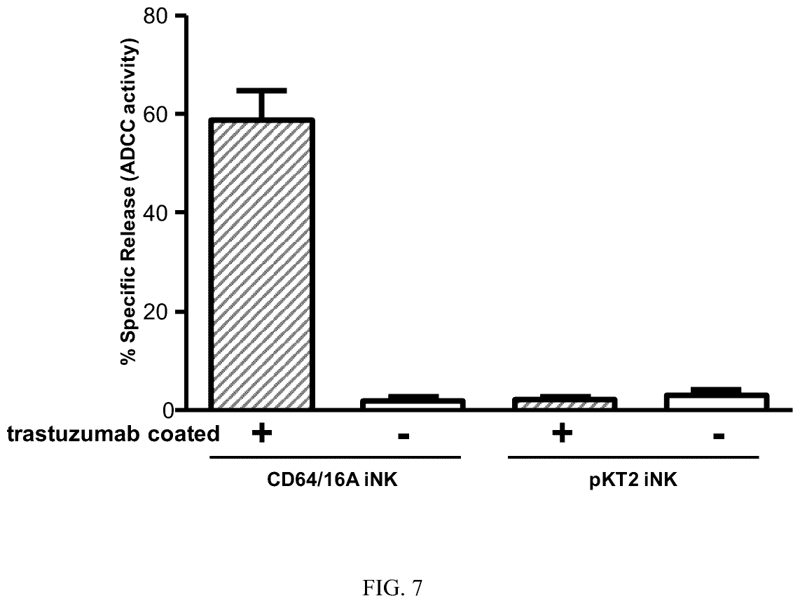

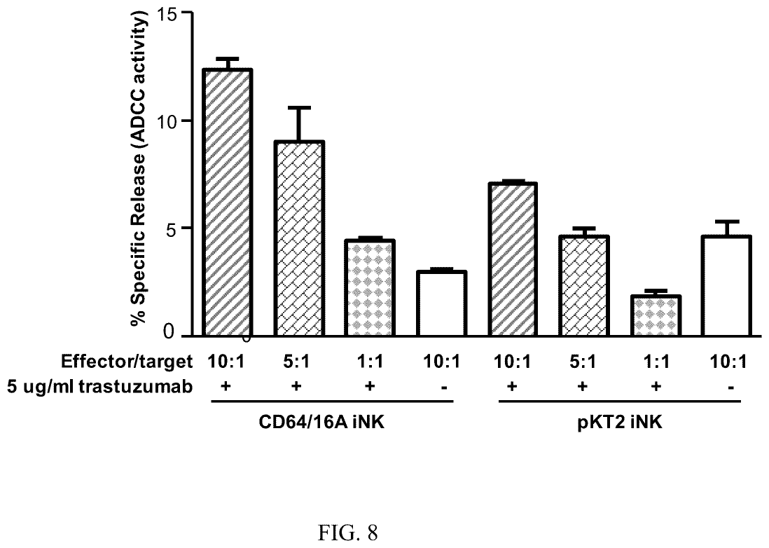

Although the iNK-CD64/16A and iNK-pKT2 cells expressed similar levels of CD16A (FIG. 5), the function blocking anti-CD16A mAb 3G8 only blocked ADCC by the iNK-pKT2 cells (FIG. 6). Conversely, the function blocking anti-CD64 mAb 10.1 only blocked ADCC by the iNK-CD64/16A cells (FIG. 6). iNK-CD64/16A and iNK-pKT2 cells were also pretreated with trastuzumab at 10 μg/ml for two hours, washed to remove unbound antibody, and then incubated with SKOV-3 cells. In this assay, iNK-CD64/16A cells demonstrated a distinct enhancement in target cell killing when compared to iNK-pKT2 cells (FIG. 7). MA148 is a human ovarian cancer cell line that expresses considerably lower levels of HER2 when compared to SKOV-3 cells. ADCC was evaluated in iNK-CD64/16A and iNK-pKT2 cells when exposed to MA148 cells at various ratios in the presence or absence of trastuzumab (5 μg/ml). Again, iNK-CD64/16A cells demonstrated significantly higher levels of tumor cell killing than did iNK-pKT2 cells at all effector to target cell ratios when in the presence of trastuzumab (FIG. 8).

The chimeric receptor CD64/16A (FIG. 2; FIG. 13A) was stably expressed in the human NK cell line NK92. These cells lack endogenous FcγRs but transduced cells expressing exogenous CD16A can mediate ADCC. As shown in FIG. 13B, an anti-CD64 mAb stained NK92 cells expressing CD64/16A cells, but not parent NK92 cells or NK92 cells expressing CD16A. An anti-CD16 mAb stained NK92 cells expressing CD16A, but not NK92 cells expressing CD64/16A or parent NK92 cells (FIG. 13B). CD16A undergoes ectodomain shedding by ADAM17 upon NK cell activation resulting in its rapid downregulation in expression. CD16A and its isoform CD16B on neutrophils is cleaved by ADAM17, and this occurs at an extracellular region proximal to the cell membrane. The ADAM17 cleavage region of CD16A is not present in CD64 or CD64/16A (FIG. 13A). CD16A underwent a >50% decrease in expression upon NK92 stimulation by ADCC, whereas CD64/16A demonstrated little to no downregulation (FIG. 13C).

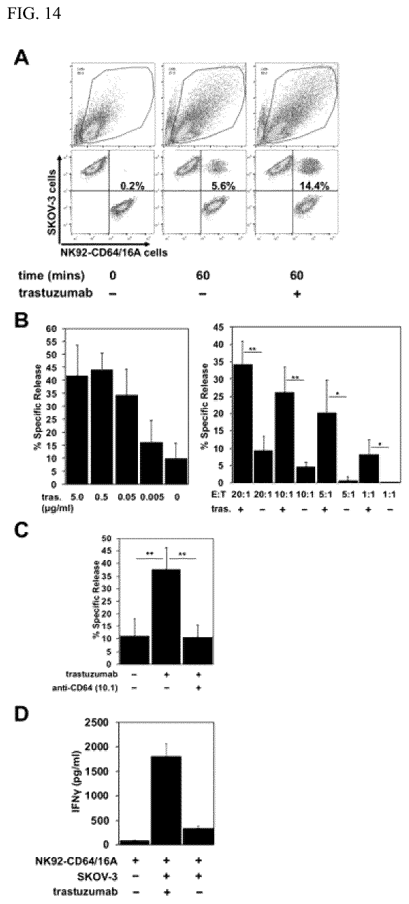

To evaluate the function of CD64/16A, the ability of CD64/16A to initiate E:T conjugation, induce ADCC, and stimulate cytokine production upon NK cell engagement of antibody-bound tumor cells was examined. Prior to the release of its granule contents, an NK cell forms a stable conjugate with a target cell. NK92-CD64/16A cell and SKOV-3 cell conjugation were examined using a two-color flow cytometric approach. SKOV-3 cells are an ovarian cancer cell line that express HER2, and this assay was performed in the absence and presence of the anti-HER2 therapeutic mAb trastuzumab. The bicistronic vector containing CD64/16A also expressed eGFP and its fluorescence was used to identify the NK92 cells. SKOV-3 cells were labeled with the fluorescent dye CellTrace Violet. E:T conjugation resulted in two-color events that were evaluated by flow cytometry. Incubating NK92-CD64/16A cells with SKOV-3 cells resulted in a very low level of conjugation after initial exposure that increased after 60 minutes of exposure (FIG. 14A). However, in the presence of trastuzumab, NK92-CD64/16A cell and SKOV-3 conjugation was appreciably enhanced (FIG. 14A). This increase in conjugation corresponded with higher levels of target cell killing. As shown in FIG. 14B, SKOV-3 cell cytotoxicity by NK92-CD64/16A cells varied depending on the trastuzumab concentration and E:T ratio. To confirm the role of CD64/16A in the induction of target cell killing, the ADCC assay was performed in the presence and absence of the anti-CD64 mAb 10.1 (FIG. 14C), which blocks IgG binding. Cytokine production is also induced during ADCC and NK cells are major producers of IFNγ. NK92-CD64/16A cells exposed to SKOV-3 cells and trastuzumab produced considerably higher levels of IFNγ than when exposed to SKOV-3 cells alone (FIG. 14D). Taken together, the above findings demonstrate that the CD64 component of the recombinant receptor engages tumor-bound antibody, and that the CD16A component promotes intracellular signaling leading to degranulation and cytokine production.

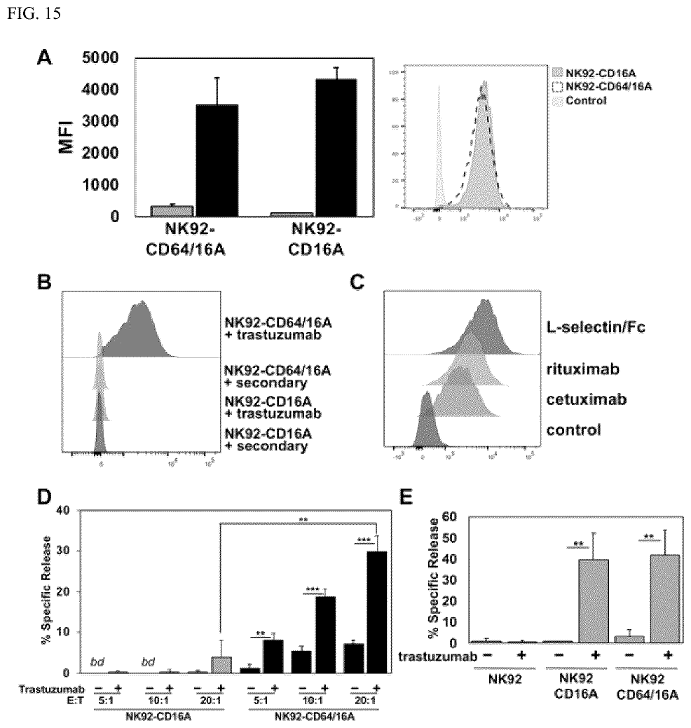

CD64 is distinguished from the other FcγR members by its unique third extracellular domain, which contributes to its high affinity and stable binding to soluble monomeric IgG. NK92 cells expressing CD64/16A or the CD16A-176V higher affinity variant were compared for their ability to capture soluble therapeutic mAbs. The NK92 cell transductants examined expressed similar levels of CD64/16A and CD16A (FIG. 15A). NK92 cell transductants were incubated with trastuzumab for two hours, excess antibody was washed away, stained with a fluorophore-conjugated anti-human IgG antibody, and then evaluated by flow cytometry. As shown in FIG. 15B, NK92-CD64/16A cells captured considerably higher levels of trastuzumab than did the NK92-CD16A cells (8.1 fold increase±1.3, mean±SD of three independent experiments). Moreover, the NK92-CD64/16A cells efficiently captured the tumor-targeting mAbs cetuximab and rituximab, as well as the fusion protein L-selectin/Fc (FIG. 15C).

NK92-CD64/16A cells with a captured tumor-targeting mAb were tested to determine whether the cells mediated ADCC. For this assay, equal numbers of NK92-CD64/16A and NK92-CD16A cells were incubated with the same concentration of soluble trastuzumab, washed, and exposed to SKOV-3 cells. Target cell killing by NK92-CD64/16A cells with captured trastuzumab was significantly higher than NK92-CD64/16A cells alone, and was superior to NK92-CD16A cells treated with or without trastuzumab at all E:T ratios examined (FIG. 15D). In contrast, SKOV-3 cytotoxicity by NK92-CD16A and NK92-CD64/16A cells was not significantly different when trastuzumab was present and not washed out (FIG. 15E), thus demonstrating equivalent cytotoxicity by both transductants. Taken together, these findings show that NK92 cells expressing CD64/16A can stably bind soluble anti-tumor mAbs and IgG fusion proteins, and that these can serve as targeting elements to kill cancer cells.

Expression and Function of CD64/16A in iPSC-Derived NK Cells

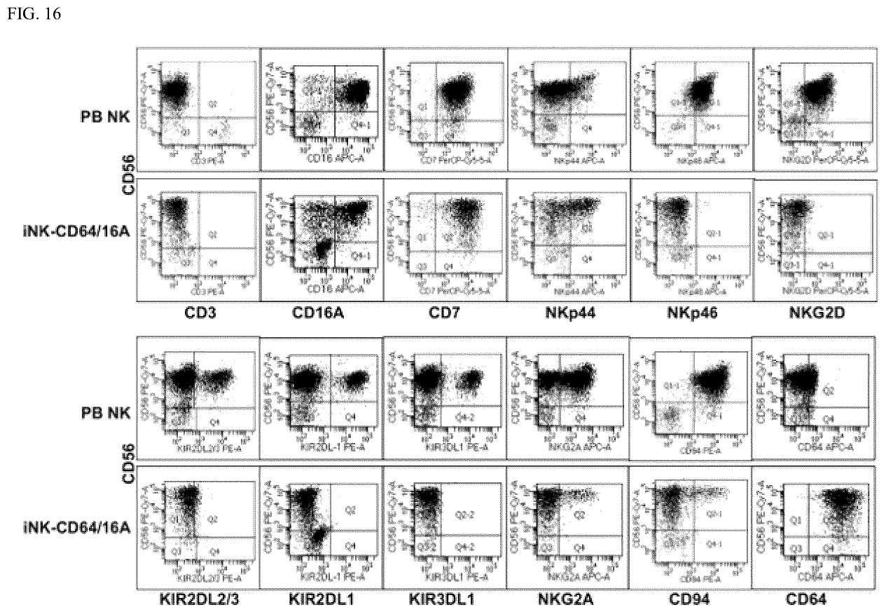

Undifferentiated iPSCs were transduced to express CD64/16A using a Sleeping Beauty transposon plasmid for nonrandom gene insertion and stable expression. iPSCs were differentiated into hematopoietic cells and then iNK cells as described in the EXAMPLE 2. CD34⁺CD43⁺CD45⁺ cells were generated, further differentiated into iNK cells, and these cells were expanded for analysis using recombinant IL-2 and aAPCs. CD56⁺CD3⁻ is a hallmark phenotype of human NK cells, and these cells composed the majority of our differentiated cell population (FIG. 16). Expression of activating and inhibitory receptors on the iNK cells also were assessed and compared to expression by peripheral blood NK cells. Certain receptors, such as CD16A, were expressed by similar proportions of the two NK cell populations. The expanded iNK cells, however, lacked expression of the inhibitory KIR receptors KIR2DL2/3, KIR2DL1, and KIR3DL1 and also certain activating receptors (NKp46 and NKG2D) (FIG. 16). Another difference compared to peripheral blood NK cells was that the iNK cells were stained with an anti-CD64 mAb (FIG. 16), demonstrating the expression of CD64/16A.

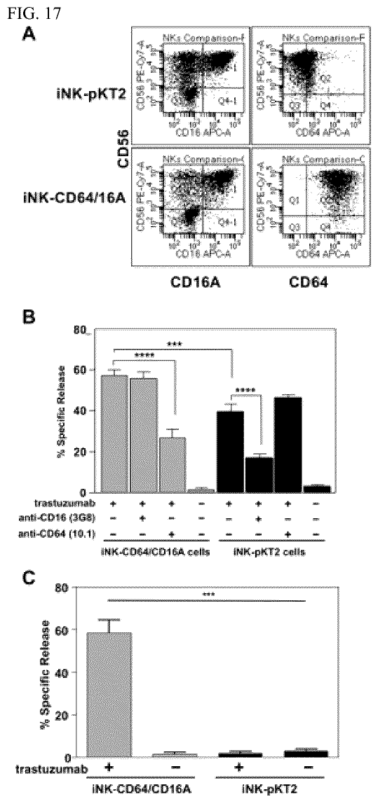

To assess the function of CD64/16A in iNK cells, iNK cells derived from iPSCs transduced with either an pKT2 empty vector or pKT2-CD64/16A were compared. The NK cell markers mentioned above were expressed at similar levels and proportions by two iNK cell populations (data not shown), including CD16A (FIG. 17A), but only iNK-CD64/16A cells were stained by an anti-CD64 mAb (FIG. 17A). Both iNK tranductants demonstrated increased SKOV-3 cell killing when in the presence of trastuzumab, yet iNK-CD64/16A cells mediated significantly higher levels of ADCC than did the iNK-pKT2 control cells (FIG. 17B). The anti-CD16 function blocking mAb 3G8, but not the anti-CD64 mAb 10.1, effectively inhibited ADCC by the iNK-pKT2 cells (FIG. 17B). Conversely, 10.1, but not 3G8, blocked ADCC by the iNK-CD64/16A cells (FIG. 17B). These findings show that the iNK cells were cytolytic effectors responsive to CD16A and CD64/16A engagement of antibody-bound tumor cells.

Also, iNK-CD64/16A and iNK-pKT2 cells were treated with soluble trastuzumab, excess antibody was washed away, and the cells were exposed to SKOV-3 cells. Under these conditions, ADCC by the iNK-CD64/16A cells was strikingly higher than the iNK-pKT2 cells (FIG. 17C), further establishing that CD64/16A can capture soluble anti-tumor mAbs that serve as a targeting element for tumor cell killing.

Taken together, the data show that CD64/16A binds therapeutic mAbs with higher affinity than CD16A. Moreover, CD64/16A expressed in NK92 cells and iNK cells confers cells with the ability to mediate higher levels of ADCC than NK92 cells and iNK cells expressing wildtype or endogenous CD16A, respectively. NK cells expressing CD64/16A facilitated cell conjugation with antibody-bound tumor cells, cytotoxicity, and IFNγ production, demonstrating function by both components of the recombinant FcγR. NK92 cells and iNK cells expressing CD64/16A can be pre-loaded with a therapeutic mAb prior to their exposure to target cells. Cells expressing the chimeric receptor and preloaded with therapeutic antibody may allow one to modify engineered NK cells with different therapeutic mAbs for targeting elements of multiple types of cancer. Finally, CD64/16A lacks the ADAM17 cleavage region found in CD16A and it did not undergo the same level of downregulation in expression during ADCC.

CD64/16A was shown to be functional in two NK cell platforms, the NK92 cell line and primary NK cells derived from iPSCs. NK92 cells lack inhibitory KIR receptors and show high levels of natural cytotoxicity compared to other NK cell lines derived from patients. NK92 cells have been broadly used to express modified genes to direct their cytolytic effector function, have been evaluated in preclinical studies, and are undergoing clinical testing in cancer patients. iPSCs are also very amendable to genetic engineering and can be differentiated into NK cells expressing various receptors to direct their effector functions. The iNK cells generated in this study lacked several inhibitory and activating receptors that are indicators of an immature state. In some applications, therapeutic iNK cells lacking inhibitory receptors and certain activating receptors may allow for more directed and/or effective tumor cell killing by engineered receptors.

The iNK cells expressed endogenous CD16A and mediated ADCC, thus they were cytotoxic effector cells. An individual NK cell can kill multiple tumor cells in different manners. This includes by a process of sequential contacts and degranulations, referred to as serial killing, and by the localized dispersion of its granule contents that kills surrounding tumor cells, referred to as bystander killing. Inhibiting CD16A shedding has been reported to slow NK cell detachment from target cells and reduce serial killing by NK cells in vitro. Due to the CD64 component and its lack of ectodomain shedding, NK cells expressing CD64/16A could be less efficient at serial killing and more efficient at bystander killing.

NK cells expressing CD64/16A have several potential advantages as a therapeutic in combination with a therapeutic antibody. Modifying NK cells expressing CD64/16A with an antibody can reduce the dosage of therapeutic antibodies administered to patients. Fusion proteins containing a human IgG Fc region, such as L-selectin/Fc, also can be captured by CD64/16A and this may provide further options for directing the tissue and tumor antigen targeting of engineered NK cells. The NK92 and iNK cell platforms for adoptive cell therapies also can be readily genetically modified on a clonal level and expanded into clinical-scalable cell numbers to produce engineered NK cells with improved effector activities as an off-the-shelf therapeutic for cancer immunotherapy.

In some embodiments, iPSC-derived NK cells that express CD64/16A can exhibit increased in vivo anti-cancer activity in the presence of therapeutic mAbs. For example, NOD/SCID/γc^{-/-} (NSG) mice and human cancer cell lines that are stably engineered to express firefly luciferase can be used for bioluminescent imaging to test iNK cell activity against cancer cells. The SKOV-3 and MA148 ovarian cancer cell line cans serve as model in vivo targets. NSG female mice can be subjected to sublethal irradiation, then injected intraperitoneally with tumor cells for bioluminescent imaging to quantify tumor growth or regression. Mice are given IL-2 and/or IL-15 every other day for four weeks to promote in vivo survival of the NK cells. Therapeutic antibody (e.g., trastuzumab) can be administered—e.g., once weekly for four weeks. Tumor growth/regression can be monitored by, for example, bioluminescent imaging and weighing the mice. Mice also can be bled (e.g., weekly) to quantify human NK cell survival, and one can further evaluate the expression/cell surface levels of various effector function markers by FACS. Evidence of metastasis can be determined by, for example, harvesting internal organs and examining the internal organs for metastasis.

In some embodiments, the number of cells in the therapeutic composition is at least 0.1×10⁵ cells, at least 1×10⁵ cells, at least 5×10⁵ cells, at least 1×10⁶ cells, at least 5×10⁶ cells, at least 1×10⁷ cells, at least 5×10⁷ cells, at least 1×10⁸ cells, at least 5×10⁸ cells, at least 1×10⁹ cells, or at least 5×10⁹ cells, per dose. In some embodiments, the number of cells in the therapeutic composition is about 0.1×10⁵ cells to about 1×10⁶ cells, per dose; about 0.5×10⁶ cells to about 1×10⁷ cells, per dose; about 0.5×10⁷ cells to about 1×10⁸ cells, per dose; about 0.5×10⁸ cells to about 1×10⁹ cells, per dose; about 1×10⁹ cells to about 5×10⁹ cells, per dose; about 0.5×10⁹ cells to about 8×10⁹ cells, per dose; about 3×10⁹ cells to about 3×10¹⁰ cells, per dose, or any range in-between. Generally, 1×10⁸ cells/dose translates to 1.67×10⁶ cells/kg for a 60 kg patient.

In one embodiment, the number of cells in the therapeutic composition is the number of immune cells in a partial or single cord of blood, or is at least 0.1×10⁵ cells/kg of bodyweight, at least 0.5×10⁵ cells/kg of bodyweight, at least 1×10⁵ cells/kg of bodyweight, at least 5×10⁵ cells/kg of bodyweight, at least 10×10⁵ cells/kg of bodyweight, at least 0.75×10⁶ cells/kg of bodyweight, at least 1.25×10⁶ cells/kg of bodyweight, at least 1.5×10⁶ cells/kg of bodyweight, at least 1.75×10⁶ cells/kg of bodyweight, at least 2×10⁶ cells/kg of bodyweight, at least 2.5×10⁶ cells/kg of bodyweight, at least 3×10⁶ cells/kg of bodyweight, at least 4×10⁶ cells/kg of bodyweight, at least 5×10⁶ cells/kg of bodyweight, at least 10×10⁶ cells/kg of bodyweight, at least 15×10⁶ cells/kg of bodyweight, at least 20×10⁶ cells/kg of bodyweight, at least 25×10⁶ cells/kg of bodyweight, at least 30×10⁶ cells/kg of bodyweight, 1×10⁸ cells/kg of bodyweight, 5×10⁸ cells/kg of bodyweight, or 1×10⁹ cells/kg of bodyweight.

In one embodiment, a dose of cells is delivered to a subject. In one illustrative embodiment, the effective amount of cells provided to a subject is at least 2×10⁶ cells/kg, at least 3×10⁶ cells/kg, at least 4×10⁶ cells/kg, at least 5×10⁶ cells/kg, at least 6×10⁶ cells/kg, at least 7×10⁶ cells/kg, at least 8×10⁶ cells/kg, at least 9×10⁶ cells/kg, or at least 10×10⁶ cells/kg, or more cells/kg, including all intervening doses of cells.

In another illustrative embodiment, the effective amount of cells provided to a subject is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, or about 10×10⁶ cells/kg, or more cells/kg, including all intervening doses of cells.

In another illustrative embodiment, the effective amount of cells provided to a subject is from about 2×10⁶ cells/kg to about 10×10⁶ cells/kg, about 3×10⁶ cells/kg to about 10×10⁶ cells/kg, about 4×10⁶ cells/kg to about 10×10⁶ cells/kg, about 5×10⁶ cells/kg to about 10×10⁶ cells/kg, 2×10⁶ cells/kg to about 6×10⁶ cells/kg, 2×10⁶ cells/kg to about 7×10⁶ cells/kg, 2×10⁶ cells/kg to about 8×10⁶ cells/kg, 3×10⁶ cells/kg to about 6×10⁶ cells/kg, 3×10⁶ cells/kg to about 7×10⁶ cells/kg, 3×10⁶ cells/kg to about 8×10⁶ cells/kg, 4×10⁶ cells/kg to about 6×10⁶ cells/kg, 4×10⁶ cells/kg to about 7×10⁶ cells/kg, 4×10⁶ cells/kg to about 8×10⁶ cells/kg, 5×10⁶ cells/kg to about 6×10⁶ cells/kg, 5×10⁶ cells/kg to about 7×10⁶ cells/kg, 5×10⁶ cells/kg to about 8×10⁶ cells/kg, or 6×10⁶ cells/kg to about 8×10⁶ cells/kg, including all intervening doses of cells.

Some variation in dosage will necessarily occur depending on the condition of the subject being treated. The person responsible for administration will, in any event, determine the appropriate dose for the individual subject.

In some embodiments, the therapeutic use of cells is a single-dose treatment. In some embodiments, the therapeutic use of derived hematopoietic lineage cells is a multi-dose treatment. In some embodiments, the multi-dose treatment is one dose every day, every 3 days, every 7 days, every 10 days, every 15 days, every 20 days, every 25 days, every 30 days, every 35 days, every 40 days, every 45 days, or every 50 days, or any number of days in-between.

A compositions that includes a population of cells described herein can be sterile, and can be suitable and ready for administration (i.e., can be administered without any further processing) to human patients. A cell-based composition that is ready for administration means that the composition does not require any further processing or manipulation prior to transplant or administration to a subject. In some embodiments, such a population of cells can include an isolated population of cells that are expanded and/or modulated prior to administration with one or more agents.

In certain embodiments, the primary stimulatory signal and the co-stimulatory signal for the therapeutic cells can be provided by different protocols. For example, the agents providing each signal can be in solution or coupled to a surface. When coupled to a surface, the agents can be coupled to the same surface (i.e., in “cis” formation) or to separate surfaces (i.e., in “trans” formation). Alternatively, one agent can be coupled to a surface and the other agent in solution. In one embodiment, the agent providing the co-stimulatory signal can be bound to a cell surface and the agent providing the primary activation signal is in solution or coupled to a surface. In certain embodiments, both agents can be in solution. In another embodiment, the agents can be in soluble form, and then cross-linked to a surface, such as a cell expressing Fc receptors or an antibody or other binding agent that will bind to the agents such as disclosed in U.S. Patent Application Publication Nos. 20040101519 and 20060034810 for artificial antigen presenting cells (aAPCs) that are contemplated for use in activating and expanding T lymphocytes.

The therapeutic compositions suitable for administration to a patient can include one or more pharmaceutically acceptable carriers (additives) and/or diluents (e.g., pharmaceutically acceptable medium, for example, cell culture medium), or other pharmaceutically acceptable components. Pharmaceutically acceptable carriers and/or diluents are determined in part by the particular composition being administered, as well as by the particular method used to administer the therapeutic composition. Accordingly, there is a wide variety of suitable formulations of therapeutic compositions (see, e.g., Remington's Pharmaceutical Sciences, 17^{th} ed. 1985, the disclosure of which is hereby incorporated by reference in its entirety) well known to those of skill in the art.

In particular embodiments, therapeutic cell compositions having an isolated population the cells as described herein also have a pharmaceutically acceptable cell culture medium, or pharmaceutically acceptable carriers and/or diluents. A therapeutic composition comprising a population of the cells as disclosed herein can be administered separately by intravenous, intraperitoneal, enteral, or tracheal administration methods or in combination with other suitable compounds to affect the desired treatment goals.

These pharmaceutically acceptable carriers and/or diluents can be present in amounts sufficient to maintain a pH of the therapeutic composition of between about 3 and about 10. As such, the buffering agent can be as much as about 5% on a weight to weight basis of the total composition. Electrolytes such as, but not limited to, sodium chloride and potassium chloride can also be included in the therapeutic composition. In one aspect, the pH of the therapeutic composition is in the range from about 4 to about 10. Alternatively, the pH of the therapeutic composition is in the range from about 5 to about 9, from about 6 to about 9, or from about 6.5 to about 8. In another embodiment, the therapeutic composition includes a buffer having a pH in one of said pH ranges. In another embodiment, the therapeutic composition has a pH of about 7. Alternatively, the therapeutic composition has a pH in a range from about 6.8 to about 7.4. In still another embodiment, the therapeutic composition has a pH of about 7.4.

This disclosure also provides the use of a pharmaceutically acceptable cell culture medium in particular compositions and/or cultures of cells as described herein. Such compositions are suitable for administration to human subjects. Generally speaking, any medium that supports the maintenance, growth, and/or health of the iPSC-derived immune cells are suitable for use as a pharmaceutical cell culture medium. In particular embodiments, the pharmaceutically acceptable cell culture medium is a serum free, and/or feeder-free medium. In various embodiments, the serum-free medium is animal-free, and can optionally be protein-free. Optionally, the medium can contain biopharmaceutically acceptable recombinant proteins. Animal-free medium refers to medium wherein the components are derived from non-animal sources. Recombinant proteins replace native animal proteins in animal-free medium and the nutrients are obtained from synthetic, plant or microbial sources. Protein-free medium, in contrast, is defined as substantially free of protein. One having ordinary skill in the art would appreciate that the above examples of media are illustrative and in no way limit the formulation of suitable media suitable and that there are many alternative suitable media known and available to those in the art.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Generation of Human CD64/16A Expression Constructs

Total RNA was isolated from peripheral blood leukocytes (PBL) using TRIzol total RNA isolation reagent (Invitrogen, Thermo Fisher Scientific, Carlsbad, CA). Human PBL cDNA was synthesized with the SuperScript Preamplification System (Invitrogen, Thermo Fisher Scientific, Carlsbad, CA). The CD64/16A includes a human CD64 extracellular domain (CD64-EC) and CD16A transmembrane and cytoplasmic domains (CD16A-TM-CY). The chimeric construct was generated by overlapping PCR to create EcoR I-flanked RT-PCR products of chimeric cDNA. CD64-EC cDNA fragment (885 bps) was amplified from human PBL cDNA using the forward primer 5′-CGG GAA TTC GGA GAC AAC ATG TGG TTC TTG ACA A-3′(SEQ ID NO:28) and reverse primer 5′-TTG GTA CCC AGG TGG AAA GAA GCC AAG CAC TTG AAG CTC CAA-3′; SEQ ID NO:29). CD16A-TM-CY cDNA fragment (195 bps) was amplified from human PBL cDNA using the forward primer 5′-TTG GAG CTT CAA GTG CTT GGC TTC TTT CCA CCT GGG TAC CAA-3′ (SEQ ID NO:30) and reverse primer 5′-CCG GAA TTC TCA TTT GTC TTG AGG GTC CTT TCT-3′ (SEQ ID NO:31). The PCR fragments of CD64-EC cDNA and CD16A-TM-CY cDNA were purified with QlAquick gel extraction kit (Qiagen, Hilden, Germany) and mixed together with the forward primer 5′-CGG GAA TTC GGA GAC AAC ATG TGG TTC TTG ACA A-3′ (SEQ ID NO:32) and the reverse primer 5′-CCG GAA TTC TCA TTT GTC TTG AGG GTC CTT TCT-3′ (SEQ ID NO:33). For all primers listed above, underlined nucleotides are the EcoR I cutting site) to generate RT-PCR products for the chimeric receptor. RT-PCR was performed with Pfx50 DNA polymerase (Invitrogen, Thermo Fisher Scientific, Carlsbad, CA). The resultant CD64/CD16a cDNA was inserted into retrovirus expression vector pBMN-IRES-EGFP (Addgene, Cambridge, MA) and pKT2 Sleeping Beauty transposon vector (Jing et al. 2015. PLoS One 10:e0121788; Hermanson et al. 2016. Stem Cells 34:93-101). The sequences of all cloned constructs were confirmed by direct sequencing from both directions on an ABI 377 sequencer with ABI BigDye terminator cycle sequencing kit (Applied Biosystems, Thermo Fisher Scientific, Foster City, CA).

### Stable Expression of CD64/16A in NK Cells

NK92 cells, a human NK cell line that is deficient for endogenous CD16A expression, were stably transduced with pBMN-IRES-EGFP retrovirus expression constructs containing CD64/16A or wildtype CD16A cDNA using retrovirus infection procedures described previously (Jing et al. 2015. PLoS One 10:e0121788). Human iPSCs (UCBiPS7, derived from umbilical cord blood CD34 cells) were stably transduced with the CD64/16A-pKT2 expression construct using a Sleeping Beauty transposon system, as previously described (Jing et al. 2015. PLoS One 10:e0121788). The transduced iPSC cells were differentiated into hematopoietic cells then mature NK cell as previously described (Jing et al. 2015. PLoS One 10:e0121788). eGFP fluorescence and surface expressions of CD64, CD16, and various NK cell phenotypic markers were determine using flow cytometry analysis.

### Example 2

### Antibodies

All mAbs to human hematopoietic and leukocyte phenotypic markers are described in Table 2. All isotype-matched negative control mAbs were purchased from BioLegend (San Diego, CA). APC-conjugated F(ab′)₂ donkey anti-human or goat anti-mouse IgG (H+L) were purchased from Jackson ImmunoResearch Laboratories (West Grove, PA). The human IgG1 mAbs trastuzumab/Herceptin and rituximab/Rituxan, manufactured by Genentech (South San Francisco, CA), and cetuximab/Erbitux, manufactured by Bristol-Myers Squibb (Lawrence, NJ), were purchased through the University of Minnesota Boynton Pharmacy. Recombinant human L-selectin/IgG1 Fc chimera was purchased from R&D Systems (Minneapolis, MN).

TABLE 2
Antibodies

| Antigen | Clone | Fluorophore | Company |
|---|---|---|---|
| CD56 | HCD56 | PE-CY7 | BioLegend, San Diego, CA |
| CD3 | HIT3a | PE | BioLegend |
| CD16 | 3G8 | APC | BioLegend |
| CD16 | 3G8 | none | Ancell, Bayport, MN |
| CD7 | CD7-6B7 | PE/CY5 | BioLegend |
| CD336/NKp44 | P44-8 | APC | BioLegend |
| CD335/NKp46 | 9E2 | APC | BioLegend |
| CD159a/NKG2A | Z199 | APC | Beckman Coulter, Brea, CA |
| CD314/NKG2D | 1D11 | PerCP/Cy5.5 | BioLegend |
| CD158a/KIR2DL1 | HP-MA4 | PE | BioLegend |
| CD158b1/KIR2DL2/L3 | DX27 | PE | BioLegend |
| CD158e1/KIR3DL1 | DX9 | PE | BioLegend |
| CD94 | DX22 | PE | BioLegend |
| CD64 | 10.1 | APC | BioLegend |
| CD64 | 10.1 | none | Ancell |
| CD34 | 561 | PE | BioLegend |
| CD45 | 2D1 | APC | BioLegend |
| CD43 | CD43-10G7 | APC | BioLegend |
| CD62L/L-selectin | LAM1-116 | APC | Ancell |

### Generation of Human CD64/16A

Total RNA was isolated from human peripheral blood leukocytes using TRIzol total RNA isolation reagent (Invitrogen, Carlsbad, CA) and cDNA was synthesized with the SuperScript preamplification system (Invitrogen, Carlsbad, CA). The recombinant CD64/16A is comprised of human CD64 extracellular domain and CD16A transmembrane and cytoplasmic domains. PCR fragments for CD64 (885 bps) and CD16A (195 bps) were amplified from the generated cDNA. The PCR fragments were purified and mixed together with the forward primer 5′-CGG GAA TTC GGA GAC AAC ATG TGG TTC TTG ACA A-3′ (SEQ ID NO:28), the reverse primer 5′-CCG GAA TTC TCA TTT GTC TTG AGG GTC CTT TCT-3′ (SEQ ID NO: 31), and Pfx50 DNA polymerase (Invitrogen) to generate the recombinant CD64/16A receptor. For both primers, underlined nucleotides are EcoR I sites. CD64/CD16A and CD16A cDNA (CD16A-176V variant) was inserted into the retroviral expression vector pBMN-IRES-EGFP and virus was generated for NK92 cell transduction, as previously described (Jing et al., 2015. PLoS One 10(3):e0121788). Additionally, CD64/CD16A cDNA was inserted into the pKT2 sleeping beauty transposon vector and used along with SB100X transposase for iPSC transduction, as previously described (Jing et al., 2015. PLoS One 10(3):e0121788). The nucleotide sequences of all constructs were confirmed by direct sequencing from both directions on an ABI 377 sequencer with ABI BigDye terminator cycle sequencing kit (Applied Biosystems, Foster City, CA).

### Cells

This study was carried out in accordance with and approved by the Institutional Review Board at the University of Minnesota. All subjects gave written informed consent in accordance with the Declaration of Helsinki. Fresh human peripheral blood leukocytes from plateletpheresis were obtained from Innovative Blood Resources (St. Paul, MN). Peripheral blood mononuclear cells were further enriched using Ficoll-Paque Plus (GE Healthcare Bio-Sciences AB, Uppsala, Sweden) and NK cells were purified by negative depletion using an EasySep human NK cell kit (StemCell Technologies, Cambridge, MA), as per the manufacturer's instructions, with >95% viability and 90-95% enrichment of CD56⁺CD3⁻ lymphocytes. Viable cell counting was performed using a Countess II automated cell counter (Life Technologies Corporation, Bothell, WA). The human NK cell line NK92 and the ovarian cancer cell line SKOV-3 were obtained from ATCC (Manassas, VA) and cultured per the manufacturer's directions. The NK92 cells required IL-2 for growth (500 IU/ml), which was obtained from R&D Systems (Minneapolis, MN). For all assays described below, cells were used when in log growth phase.

The iPSCs UCBiPS7, derived from umbilical cord blood CD34 cells, have been previously characterized and were cultured and differentiated into hematopoietic progenitor cells as described with some modifications (Jing et al., 2015. PLoS One 10:e0121788; Ni et al., 2013. Methods in molecular biology 1029:33-41; Knorr et al., 2013. Stem Cells Transl Med 2(4):274-283; Ni et al., 2014. Stem Cells 32(4):1021-1031; Hermanson et al., 2016. Stem Cells 34(1):93-101). iPSCs culture and hematopoietic differentiation was performed using TeSR-E8 medium and a STEMdiff Hematopoietic Kit (StemCell Technologies, Cambridge, MA), which did not require the use of mouse embryonic fibroblast feeder cells, TrypLE adaption, spin embryoid body formation, or CD34⁺ cell enrichment. iPSC cells during passage were dissociated with Gentle Cell Dissociation Reagent (StemCell Technologies, Cambridge, MA), and iPSC aggregates ≥50 μm in diameter were counted with a hemocytometer and diluted to 20-40 aggregates/ml with TeSR-E8 medium. Each well of a 12-well plate was pre-coated with Matrigel Matrix (Corning Inc., Tewksbury, MA) and seeded with 40-80 aggregates in 2 ml of TeSR-E8 medium. Cell aggregates were cultured for 24 hours before differentiation with the STEMdiff Hematopoietic Kit (StemCell Technologies, Cambridge, MA) according to the manufacturer's instructions. At day 12 of hematopoietic progenitor cell differentiation, the percentage of hematopoietic progenitor cells was determined using flow cytometric analysis with anti-CD34, anti-CD45, and anti-CD43 mAbs. NK cell differentiation was performed as previously described (Woll et al., 2009. Blood 113(24):6094-6101). The iPSC-derived NK cells (referred to here as iNK cells) were expanded for examination using γ-irradiated K562-mbIL21-41BBL feeder cells (1:2 ratio) in cell expansion medium [60% DMEM, 30% Ham's F12, 10% human AB serum (Valley Biomedical, Winchester, VA), 20 μM 2-mercaptoethanol, 50 μM ethanolamine, 20 μg/ml ascorbic Acid, 5 ng/ml sodium selenite, 10 mM HEPES, and 100-250 IU/ml IL-2 (R&D Systems)], as described previously (Jing et al., 2015. PLoS One 10:e0121788; Knorr et al., 2013. Stem Cells Transl Med 2(4):274-283; Ni et al., 2014. Stem Cells 32(4):1021-1031; Hermanson et al., 2016. Stem Cells 34(1):93-101).

### Cell Staining, Flow Cytometry, and ELISA

For cell staining, nonspecific antibody binding sites were blocked and cells were stained with the indicated antibodies and examined by flow cytometry, as previously described (Romee et al, 2013. Blood 121(18):3599-3608; Jing et al., 2015. PLoS One 10:e0121788; Mishra et al., 2018. Cancer Immunol Immunother 67(9):1407-1416). For controls, fluorescence minus one was used as well as appropriate isotype-matched antibodies since the cells of interest expressed FcRs. An FSC-A/SSC-A plot was used to set an electronic gate on leukocyte populations and an FSC-A/FSC-H plot was used to set an electronic gate on single cells. A Zombie viability kit was used to assess live vs. dead cells, as per the manufacturer's instructions (BioLegend, San Diego, CA).

To assess the capture of soluble trastuzumab, rituximab, cetuximab, or L-selectin/Fc chimera, transduced NK cells were incubated with 5 μg/ml of antibody for two hours at 37° C. in MEM-α basal media (Thermo Fisher Scientific, Waltham, MA) supplemented with IL-2 (200 IU/ml), HEPES (10 mM), and 2-mercaptoethanol (0.1 mM), washed with MEM-α basal media, and then stained on ice for 30 minutes with a 1:200 dilution of APC-conjugated F(ab′)₂ donkey anti-human IgG (H+L). To detect recombinant human L-selectin/Fc binding, cells were stained with the anti-L-selectin mAb APC-conjugated Lam1-116.

To compare CD16A, CD64, and CD64/16A staining levels on NK92 cells, the respective transductants were stained with a saturating concentration of unconjugated anti-CD16 (3G8) or anti-CD64 (10.1) mAbs (5 μg/ml), washed extensively with dPBS (USB Corporation, Cleveland, OH) containing 2% goat serum and 2 mM sodium azide, and then stained with APC-conjugated F(ab′)₂ goat anti-mouse IgG (H+L). This approach was used since directly conjugated anti-CD16 and anti-CD64 mAbs can vary in their levels of fluorophore labeling. ELISA was performed by a cytometric bead-based Flex Set assay for human IFNγ (BD Biosciences, San Jose, CA) per the manufacturer's instructions. All flow cytometric analyses were performed on a FACSCelesta instrument (BD Biosciences). Data was analyzed using FACSDIVA v8 (BD Biosciences) and FlowJo v10 (Ashland, OR).

### Cell-Cell Conjugation Assay and ADCC

The pBMN-IRES-EGFP vector used to express CD64/16A in NK92 cells also expresses eGFP. These cells were incubated for two hours at 37° C. in MEM-α basal media (Thermo Fisher Scientific, Waltham, MA) supplemented with IL-2 (200 IU/ml), HEPES (10 mM), and 2-mercaptoethanol (0.1 mM). SKOV-3 cells were labeled with CellTrace Violet (Molecular Probes, Eugene, OR) per the manufacturer's instructions, incubated with 5 μg/ml trastuzumab for 30 minutes and washed with the MEM-α basal media. NK92 cells and SKOV-3 cells were then resuspended in the supplemented MEM-α basal media at 1×10⁶ and 2×10⁶/ml, respectively. For a 1:2 E:T ratio, 100 μl of each cell type was mixed together, centrifuged for one minute at 20×g and incubated at 37° C. for the indicated time points. After each time point, the cells were gently vortexed for three seconds and immediately fixed with 4° C. 1% paraformaldehyde in dPBS. Samples were immediately analyzed by flow cytometry. The percentage of conjugated NK cells was calculated by gating on eGFP and CellTrace Violet double-positive events.

To evaluate ADCC, a DELFIA EuTDA-based cytotoxicity assay was used according to the manufacturer's instructions (PerkinElmer, Waltham, MA). Briefly, target cells were labeled with Bis(acetoxymethyl)-2-2:6,2 terpyridine 6,6 dicarboxylate (BATDA) for 30 minutes in their culture medium, washed in culture medium, and pipetted into a 96-well non-tissue culture-treated U-bottom plates at a density of 8×10⁴ cells/well. A tumor targeting mAb was added at the indicated concentrations of 5 μg/mL and NK cells were added at the indicated effector:target (E:T) ratios. The plates were centrifuged at 400×g for one minute and then incubated for two hours in a humidified 5% CO₂ atmosphere at 37° C. At the end of the incubation, the plates were centrifuged at 500×g for five minutes and supernatants were transferred to a 96-well DELFIA Yellow Plate (PerkinElmer, Waltham, MA) and combined with europium. Fluorescence was measured by time-resolved fluorometry using a BMG Labtech CLARIOstar plate reader (Cary, NC). BATDA-labeled target cells alone with or without therapeutic antibodies were cultured in parallel to assess spontaneous lysis and in the presence of 2% Triton-X to measure maximum lysis. The level of ADCC for each sample was calculated using the formula: Percent Specific Release=(Experimental release counts−Spontaneous release counts)/(Maximal release−Spontaneous release counts)×100%. For each experiment, measurements were conducted in triplicate using three replicate wells.

### Statistical Analyses

Statistical analyses were performed by use of GraphPad Prism (GraphPad Software, La Jolla, CA, USA). After assessing for approximate normal distribution, all variables were summarized as mean±SD. Comparison between two groups was done with Student's t-test, with p<0.05 taken as statistically significant.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

SEQUENCE LISTING FREE TEXT
rCD64#1 (pCDH-CD64) peptide sequence
(SEQ ID NO: 9)
MWFLTTLLLW VPVDGQVDTT KAVITLQPPW VSVFQEETVT
LHCEVLHLPG SSSTQWFLNG TATQTSTPSY RITSASVNDS
GEYRCQRGLS GRSDPIQLEI HRGWLLLQVS SRVFTEGEPL
ALRCHAWKDK LVYNVLYYRN GKAFKFFHWN SNLTILKTNI
SHNGTYHCSG MGKHRYTSAG ISVTVKELFP APVLNASVTS
PLLEGNLVTL SCETKLLLQR PGLQLYFSFY MGSKTLRGRN
TSSEYQILTA RREDSGLYWC EAATEDGNVL KRSPELELQV
LGLQLPTPVW FHVLFYLAVG IMFLVNTVLW VTIRKELKRK
KKWDLEISLD SGHEKKVISS LQEDRHLEEE LKCQEQKEEQ
LQEGVHRKEP QGAT
rCD64#2 (pCDH-mutCD64) peptide sequence
(SEQ ID NO: 10)
MWFLTTLLLW VPVDGQVDTT KAVITLQPPW VSVFQEETVT
LHCEVLHLPG SSSTQWFLNG TATQTSTPSY RITSASVNDS
GEYRCQRGLS GRSDPIQLEI HRGWLLLQVS SRVFTEGEPL
ALRCHAWKDK LVYNVLYYRN GKAFKFFHWN SNLTILKTNI
SHNGTYHCSG MGKHRYTSAG ISVTVKELFP APVLNASVTS
PLLEGNLVTL SCETKLLLQR PGLQLYFSFY MGSKTLRGRN
TSSEYQILTA RREDSGLYWC EAATEDGNVL KRSPELELQV
LGLQLPTPVW FHVLFYLAVG IMFLVNTVLW VTIRKELKRK
KKWNLEISLD SGHEKKVISS LQEDRHLEEE LKCQEQKEEQ
LQEGVHRKEP QGAT
rCD64#3 (pCDH-CD64/16) peptide sequence
(SEQ ID NO: 11)
MWFLTTLLLW VPVDGQVDTT KAVITLQPPW VSVFQEETVT
LHCEVLHLPG SSSTQWFLNG TATQTSTPSY RITSASVNDS
GEYRCQRGLS GRSDPIQLEI HRGWLLLQVS SRVFTEGEPL
ALRCHAWKDK LVYNVLYYRN GKAFKFFHWN SNLTILKTNI
SHNGTYHCSG MGKHRYTSAG ISVTVKELFP APVLNASVTS
PLLEGNLVTL SCETKLLLQR PGLQLYFSFY MGSKTLRGRN
TSSEYQILTA RREDSGLYWC EAATEDGNVL KRSPELELQV
LGFFPPGYQV SFCLVMVLLF AVDTGLYFSV KTNIRSSTRD
WKDHKFKWRK DPQDK
rCD64#4 (pCDH-CD64/16-28-BB-Z) peptide sequence
(SEQ ID NO: 12)
MWFLTTLLLW VPVDGQVDTT KAVISLQPPW VSVFQEETVT
LHCEVLHLPG SSSTQWFLNG TATQTSTPSY RITSASVNDS
GEYRCQRGLS GRSDPIQLEI HRGWLLLQVS SRVFTEGEPL
ALRCHAWKDK LVYNVLYYRN GKAFKFFHWN SNLTILKTNI
SHNGTYHCSG MGKHRYTSAG ISVTVKELFP APVLNASVTS
PLLEGNLVTL SCETKLLLQR PGLQLYFSFY MGSKTLRGRN
TSSEYQILTA RREDSGLYWC EAATEDGNVL KRSPELELQV
LGFFPPGYQV SFCLVMVLLF AVDTGLYFSV KTNIRSSTRD
WKDHKFKWRK DPQDKRSKRS RLLHSDYMNM TPRRPGPTRK
HYQPYAPPRD FAAYRSKRGR KKLLYIFKQP FMRPVQTTQE
EDGCSCRFPE EEEGGCELRV KFSRSADAPA YQQGQNQLYN
ELNLGRREEY DVLDKRRGRD PEMGGKPRRK NPQEGLYNEL
QKDKMAEAYS EIGMKGERRR GKGHDGLYQG LSTATKDTYD
ALHMQALPPR
rCD64#5 (pCDH-CD64/16-28-BB-G) peptide sequence
(SEQ ID NO: 13)
MWFLTTLLLW VPVDGQVDTT KAVITLQPPW VSVFQEETVT
LHCEVLHLPG SSSTQWFLNG TATQTSTPSY RITSASVNDS
GEYRCQRGLS GRSDPIQLEI HRGWLLLQVS SRVFTEGEPL
ALRCHAWKDK LVYNVLYYRN GKAFKFFHWN SNLTILKTNI
SHNGTYHCSG MGKHRYTSAG ISVTVKELFP APVLNASVTS
PLLEGNLVTL SCETKLLLQR PGLQLYFSFY MGSKTLRGRN
TSSEYQILTA RREDSGLYWC EAATEDGNVL KRSPELELQV
LGFFPPGYQV SFCLVMVLLF AVDTGLYFSV KTNIRSSTRD
WKDHKFKWRK DPQDKRSKRS RLLHSDYMNM TPRRPGPTRK
HYQPYAPPRD FAAYRSKRGR KKLLYIFKQP FMRPVQTTQE
EDGCSCRFPE EEEGGCELRL KIQVRKAAIT SYEKSDGVYT
GLSTRNQETY ETLKHEKPPQ
rCD64#6 (pCDH-CD64/16-28-BB-D10) peptide sequence
(SEQ ID NO: 14)
MWFLTTLLLW VPVDGQVDTT KAVITLQPPW VSVFQEETVT
LHCEVLHLPG SSSTQWFLNG TATQTSTPSY RITSASVNDS
GEYRCQRGLS GRSDPIQLEI HRGWLLLQVS SRVFTEGEPL
ALRCHAWKDK LVYNVLYYRN GKAFKFFHWN SNLTILKTNI
SHNGTYHCSG MGKHRYTSAG ISVTVKELFP APVLNASVTS
PLLEGNLVTL SCETKLLLQR PGLQLYFSFY MGSKTLRGRN
TSSEYQILTA RREDSGLYWC EAATEDGNVL KRSPELELQV
LGFFPPGYQV SFCLVMVLLF AVDTGLYFSV KTNIRSSTRD
WKDHKFKWRK DPQDKRSKRS RLLHSDYMNM TPRRPGPTRK
HYQPYAPPRD FAAYRSKRGR KKLLYIFKQP FMRPVQTTQE
EDGCSCRFPE EEEGGCELAR PRRSPAQEDG KVYINMPGRG
rCD64#7 (pCDH-CD64/16-28-BB-D12) peptide sequence
(SEQ ID NO: 15)
MWFLTTLLLW VPVDGQVDTT KAVITLQPPW VSVFQEETVT
LHCEVLHLPG SSSTQWFLNG TATQTSTPSY RITSASVNDS
GEYRCQRGLS GRSDPIQLEI HRGWLLLQVS SRVFTEGEPL
ALRCHAWKDK LVYNVLYYRN GKAFKFFHWN SNLTILKTNI
SHNGTYHCSG MGKHRYTSAG ISVTVKELFP APVLNASVTS
PLLEGNLVTL SCETKLLLQR PGLQLYFSFY MGSKTLRGRN
TSSEYQILTA RREDSGLYWC EAATEDGNVL KRSPELELQV
LGFFPPGYQV SFCLVMVLLF AVDTGLYFSV KTNIRSSTRD
WKDHKFKWRK DPQDKRSKRS RLLHSDYMNM TPRRPGPTRK
HYQPYAPPRD FAAYRSKRGR KKLLYIFKQP FMRPVQTTQE
EDGCSCRFPE EEEGGCELGR LVPRGRGAAE AATRKQRITE
TESPYQELQG QRSDVYSDLN TQRPYYK
rCD64#8 (pCDH-CD64-28-28-BB-Z) peptide sequence
(SEQ ID NO: 16)
MWFLTTLLLW VPVDGQVDTT KAVITLQPPW VSVFQEETVT
LHCEVLHLPG SSSTQWFLNG TATQTSTPSY RITSASVNDS
GEYRCQRGLS GRSDPIQLEI HRGWLLLQVS SRVFTEGEPL
ALRCHAWKDK LVYNVLYYRN GKAFKFFHWN SNLTILKTNI
SHNGTYHCSG MGKHRYTSAG ISVTVKELFP APVLNASVTS
PLLEGNLVTL SCETKLLLQR PGLQLYFSFY MGSKTLRGRN
TSSEYQILTA RREDSGLYWC EAATEDGNVL KRSPELELQV
LGLQLPTPFW VLVVVGGVLA CYSLLVTVAF IIFWVRSKRS
RLLHSDYMNM TPRRPGPTRK HYQPYAPPRD FAAYRSKRGR
KKLLYIFKQP FMRPVQTTQE EDGCSCRFPE EEEGGCELRV
KFSRSADAPA YQQGQNQLYN ELNLGRREEY DVLDKRRGRD
PEMGGKPRRK NPQEGLYNEL QKDKMAEAYS EIGMKGERRR
GKGHDGLYQG LSTATKDTYD ALHMQALPPR
rCD64#9 (pCDH-CD64-FceR) peptide sequence
(SEQ ID NO: 17)
MWFLTTLLLW VPVDGQVDTT KAVITLQPPW VSVFQEETVT
LHCEVLHLPG SSSTQWFLNG TATQTSTPSY RITSASVNDS
GEYRCQRGLS GRSDPIQLEI HRGWLLLQVS SRVFTEGEPL
ALRCHAWKDK LVYNVLYYRN GKAFKFFHWN SNLTILKTNI
SHNGTYHCSG MGKHRYTSAG ISVTVKELFP APVLNASVTS
PLLEGNLVTL SCETKLLLQR PGLQLYFSFY MGSKTLRGRN
TSSEYQILTA RREDSGLYWC EAATEDGNVL KRSPELELQV
LGAPREKYWL QFFIPLLVVI LFAVDTGLFI STQQQVTFLL
KIKRTRKGFR LLNPHPKPNP KNN
rCD64#10 (pCDH-CD64/16-PKC⁻) peptide sequence
(SEQ ID NO: 18)
MWFLTTLLLW VPVDGQVDTT KAVITLQPPW VSVFQEETVT
LHCEVLHLPG SSSTQWFLNG TATQTSTPSY RITSASVNDS
GEYRCQRGLS GRSDPIQLEI HRGWLLLQVS SRVFTEGEPL
ALRCHAWKDK LVYNVLYYRN GKAFKFFHWN SNLTILKTNI
SHNGTYHCSG MGKHRYTSAG ISVTVKELFP APVLNASVTS
PLLEGNLVTL SCETKLLLQR PGLQLYFSFY MGSKTLRGRN
TSSEYQILTA RREDSGLYWC EAATEDGNVL KRSPELELQV
LGFFPPGYQV SFCLVMVLLF AVDTGLYFSV KTNIRGAGRD
WKDHKFKWRK DPQDK
rCD64#11 (pCDH-CD64-G2D-2B4-Z) peptide sequence
(SEQ ID NO: 19)
MWFLTTLLLW VPVDGQVDTT KAVITLQPPW VSVFQEETVT
LHCEVLHLPG SSSTQWFLNG TATQTSTPSY RITSASVNDS
GEYRCQRGLS GRSDPIQLEI HRGWLLLQVS SRVFTEGEPL
ALRCHAWKDK LVYNVLYYRN GKAFKFFHWN SNLTILKTNI
SHNGTYHCSG MGKHRYTSAG ISVTVKELFP APVLNASVTS
PLLEGNLVTL SCETKLLLQR PGLQLYFSFY MGSKTLRGRN
TSSEYQILTA RREDSGLYWC EAATEDGNVL KRSPELELQV
LGSNLFVASW IAVMIIFRIG MAVAIFCCFF FPSGGSGGGS
GWRRKRKEKQ SETSPKEFLT IYEDVKDLKT RRNHEQEQTF
PGGGSTIYSM IQSQSSAPTS QEPAYTLYSL IQPSRKSGSR
KRNHSPSFNS TIYEVIGKSQ PKAQNPARLS RKELENFDVY
SGGSGGGSGR VKFSRSADAP AYKQGQNQLY NELNLGRREE
YDVLDKRRGR DPEMGGKPRR KNPQEGLYNE LQKDKMAEAY
SEIGMKGERR RGKGHDGLYQ GLSTATKDTY DALHMQALPP
R
rCD64#12 (pCDH-CD64/16-2B4-Z) peptide sequence
(SEQ ID NO: 20)
MWFLTTLLLW VPVDGQVDTT KAVITLQPPW VSVFQEETVT
LHCEVLHLPG SSSTQWFLNG TATQTSTPSY RITSASVNDS
GEYRCQRGLS GRSDPIQLEI HRGWLLLQVS SRVFTEGEPL
ALRCHAWKDK LVYNVLYYRN GKAFKFFHWN SNLTILKTNI
SHNGTYHCSG MGKHRYTSAG ISVTVKELFP APVLNASVTS
PLLEGNLVTL SCETKLLLQR PGLQLYFSFY MGSKTLRGRN
TSSEYQILTA RREDSGLYWC EAATEDGNVL KRSPELELQV
LGFFPPGYQV SFCLVMVLLF AVDTGLYFSV KTNIRSSTRD
WKDHKFKWRK DPQDKWRRKR KEKQSETSPK EFLTIYEDVK
DLKTRRNHEQ EQTFPGGGST IYSMIQSQSS APTSQEPAYT
LYSLIQPSRK SGSRKRNHSP SFNSTIYEVI GKSQPKAQNP
ARLSRKELEN FDVYSGGSGG GSGRVKFSRS ADAPAYKQGQ
NQLYNELNLG RREEYDVLDK RRGRDPEMGG KPRRKNPQEG
LYNELQKDKM AEAYSEIGMK GERRRGKGHD GLYQGLSTAT
KDTYDALHMQ ALPPR
rCD64#13 (pCDH-CD64/16-2B4-G) peptide sequence
(SEQ ID NO: 21)
MWFLTTLLLW VPVDGQVDTT KAVITLQPPW VSVFQEETVT
LHCEVLHLPG SSSTQWFLNG TATQTSTPSY RITSASVNDS
GEYRCQRGLS GRSDPIQLEI HRGWLLLQVS SRVFTEGEPL
ALRCHAWKDK LVYNVLYYRN GKAFKFFHWN SNLTILKTNI
SHNGTYHCSG MGKHRYTSAG ISVTVKELFP APVLNASVTS
PLLEGNLVTL SCETKLLLQR PGLQLYFSFY MGSKTLRGRN
TSSEYQILTA RREDSGLYWC EAATEDGNVL KRSPELELQV
LGFFPPGYQV SFCLVMVLLF AVDTGLYFSV KTNIRSSTRD
WKDHKFKWRK DPQDKWRRKR KEKQSETSPK EFLTIYEDVK
DLKTRRNHEQ EQTFPGGGST IYSMIQSQSS APTSQEPAYT
LYSLIQPSRK SGSRKRNHSP SFNSTIYEVI GKSQPKAQNP
ARLSRKELEN FDVYSGGSGG GSGRLKIQVR KAAITSYEKS
DGVYTGLSTR NQETYETLKH EKPPQ
rCD64#14 (pCDH-CD64/16-2B4-D10) peptide sequence
(SEQ ID NO: 22)
MWFLTTLLLW VPVDGQVDTT KAVITLQPPW VSVFQEETVT
LHCEVLHLPG SSSTQWFLNG TATQTSTPSY RITSASVNDS
GEYRCQRGLS GRSDPIQLEI HRGWLLLQVS SRVFTEGEPL
ALRCHAWKDK LVYNVLYYRN GKAFKFFHWN SNLTILKTNI
SHNGTYHCSG MGKHRYTSAG ISVTVKELFP APVLNASVTS
PLLEGNLVTL SCETKLLLQR PGLQLYFSFY MGSKTLRGRN
TSSEYQILTA RREDSGLYWC EAATEDGNVL KRSPELELQV
LGFFPPGYQV SFCLVMVLLF AVDTGLYFSV KTNIRSSTRD
WKDHKFKWRK DPQDKWRRKR KEKQSETSPK EFLTIYEDVK
DLKTRRNHEQ EQTFPGGGST IYSMIQSQSS APTSQEPAYT
LYSLIQPSRK SGSRKRNHSP SFNSTIYEVI GKSQPKAQNP
ARLSRKELEN FDVYSGGSGG GSGARPRRSP AQEDGKVYIN
MPGRG
rCD64#15 (pCDH-CD64/16-2B4-D12) peptide sequence
(SEQ ID NO: 23)
MWFLTTLLLW VPVDGQVDTT KAVITLQPPW VSVFQEETVT
LHCEVLHLPG SSSTQWFLNG TATQTSTPSY RITSASVNDS
GEYRCQRGLS GRSDPIQLEI HRGWLLLQVS SRVFTEGEPL
ALRCHAWKDK LVYNVLYYRN GKAFKFFHWN SNLTILKTNI
SHNGTYHCSG MGKHRYTSAG ISVTVKELFP APVLNASVTS
PLLEGNLVTL SCETKLLLQR PGLQLYFSFY MGSKTLRGRN
TSSEYQILTA RREDSGLYWC EAATEDGNVL KRSPELELQV
LGFFPPGYQV SFCLVMVLLF AVDTGLYFSV KTNIRSSTRD
DPQDKWRRKR KEKQSETSPK WKDHKFKWRK EFLTIYEDVK
DLKTRRNHEQ EQTFPGGGST IYSMIQSQSS APTSQEPAYT
LYSLIQPSRK SGSRKRNHSP SFNSTIYEVI GKSQPKAQNP
ARLSRKELEN FDVYSGGSGG GSGGRLVPRG RGAAEAATRK
QRITETESPY QELQGQRSDV YSDLNTQRPY YK
rCD64#16 (pCDH-CD64-64-2B4-Z) peptide sequence
(SEQ ID NO: 24)
MWFLTTLLLW VPVDGQVDTT KAVITLQPPW VSVFQEETVT
LHCEVLHLPG SSSTQWFLNG TATQTSTPSY RITSASVNDS
GEYRCQRGLS GRSDPIQLEI HRGWLLLQVS SRVFTEGEPL
ALRCHAWKDK LVYNVLYYRN GKAFKFFHWN SNLTILKTNI
SHNGTYHCSG MGKHRYTSAG ISVTVKELFP APVLNASVTS
PLLEGNLVTL SCETKLLLQR PGLQLYFSFY MGSKTLRGRN
TSSEYQILTA RREDSGLYWC EAATEDGNVL KRSPELELQV
LGLQLPTPVW FHVLFYLAVG IMFLVNTVLW VTIRKELKRK
KKWDLEISLD SGHEKKVISS LQEDRHLEEE LKCQEQKEEQ
LQEGVHRKEP QGATGWRRKR KEKQSETSPK EFLTIYEDVK
DLKTRRNHEQ EQTFPGGGST IYSMIQSQSS APTSQEPAYT
LYSLIQPSRK SGSRKRNHSP SFNSTIYEVI GKSQPKAQNP
ARLSRKELEN FDVYSGGSGG GSGRVKFSRS ADAPAYKQGQ
NQLYNELNLG RREEYDVLDK RRGRDPEMGG KPRRKNPQEG
LYNELQKDKM AEAYSEIGMK GERRRGKGHD GLYQGLSTAT
KDTYDALHMQ ALPPR
Canine CD16#17 (pCDH-caCD16) peptide sequence
(SEQ ID NO: 25)
MWQLVSSTAL LLLVSAGTQA DVPKAVVVLE PKWNRVLTMD
SVTLKCQGDH LLRDNYTWLH NGRPISNQIS TYIIKNASIK
NSGEYRCQTD QSKLSDPVQL EVHTGWLLLQ VPRLVFQEGE
LIQLKCHSWK NTPVRNVQYF QNGRGKKFFY NNSEYHIPAA
TSEHNGSYFC RGIIGKKNES SEAVNIIIQG SSLPSTSLLL
SHWPQIPFSL VMALLFAVDT GLYFAVQRDL RSSMGNLKNS
KVIWSQGS
Canine CD64#18 (pCDH-caCD64) peptide sequence
(SEQ ID NO: 26)
MWLLTVLLLW VPAGAQTDPV KAVITLQPPW VSVFQEESVT
LWCEGPHLPG DSSTQWFLNG TATQTLTPRY RIAAASVNDN
GEYRCQTGLS VLSDPIQLGI HRDWLILQVS GRVFTEGEPL
TLRCHGWNNK LVYNVLFYQN GTVLKFSPQN SEFTILKTTL
HHNGIYHCSA MGKHRYESAG VSITIKELFP APVLKASLSS
PILEGHVVNL SCETKLLLQR PGLQLYFSFY MGSKTLLSRN
TSSEYQILTA KKEDSGLYWC EATTEDGNVV KRSPELELQV
VGPQTLTPVW FHVLFYVAMG MIFLVDTIFC MIIHKELQRK
KKWNLEISLY SGLEKRVDSY LQKERDLEEP KYQELEQLQE
KTPQKPPEGE QQ
Canine CD64sp#19 (pCDH-caCD64sp) peptide sequence
(SEQ ID NO: 27)
MWLLTVLLLW VPAGAQTDWL ILQVSGRVFT EGEPLTLRCH
GWNNKLVYNV LFYQNGTVLK FSPQNSEFTI LKTTLHHNGI
YHCSAMGKHR YESAGVSITI KELFPAPVLK ASLSSPILEG
HVVNLSCETK LLLQRPGLQL YFSFYMGSKT LLSRNTSSEY
QILTAKKEDS GLYWCEATTE DGNVVKRSPE LELQVVGPQT
LTPVWFHVLF YVAMGMIFLV DTIFCMIIHK ELQRKKKWNL
EISLYSGLEK RVDSYLQKER DLEEPKYQEL EQLQEKTPQK
PPEGEQQ

1-6. (canceled)

7. A recombinant cell comprising a polynucleotide encoding a chimeric IgG Fc receptor, wherein the recombinant cell is an induced pluripotent stem cell (iPSC), and further wherein the chimeric IgG Fc receptor comprises:
(i) an extracellular domain comprising a CD64 extracellular domain that binds to an IgG Fc region;
(ii) a transmembrane domain; and
(iii) an intracellular domain comprising an Fc receptor immunoreceptor tyrosine-based activation motif (ITAM).

8. The recombinant cell of claim 7, wherein the recombinant cell expresses the chimeric IgG Fc receptor.

9. The recombinant cell of claim 7, wherein the iPSC is capable of differentiating into is a natural killer (NK) cell.

10. (canceled)

11. The recombinant cell of claim 9, wherein the natural killer (NK) cell expresses the chimeric IgG Fc receptor.

12. A method of killing a tumor cell, the method comprising:
differentiating the iPSC of claim 7 to produce a derivative NK cell expressing the chimeric IgG Fc receptor;
contacting the tumor cell with an antibody; and
contacting the tumor cell with the derivative NK cell under conditions effective for the derivative NK cell to kill the tumor cell.

13. A method of treating a subject having a tumor, the method comprising:
differentiating the iPSC of claim 7 to produce a derivative NK cell expressing the chimeric IgG Fc receptor; and
administering to the subject a composition comprising the derivative NK.

14. A composition comprising:
a cell differentiated from the iPSC of claim 7; and
an antibody bound to the chimeric IgG Fc receptor.

15. A method of treating a subject having a tumor, the method comprising:
administering to the subject the composition of claim 14.

16. The method of claim 15, wherein the method further comprises forming a complex comprising the recombinant cell differentiated from the iPSC, the chimeric IgG Fc receptor, the antibody, and a tumor cell.

17. A method of producing a recombinant iPSC-derived cell, the method comprising differentiating the recombinant cell of claim 7 to produce the iPSC-derived cell.

18. The method of claim 17, wherein the iPSC-derived cell is an NK cell.

19. The method of claim 18, wherein the NK cell expresses the chimeric IgG Fc receptor.

20. The recombinant cell of claim 7, wherein the ITAM is effective to initiate cell signaling in response to binding of an IgG Fc region to the extracellular domain.

21. The recombinant cell of claim 7, wherein the intracellular domain comprises an intracellular domain of CD16A.

22. The recombinant cell of claim 7, wherein the intracellular domain comprises an intracellular domain of CD27, CD28, CD134 (OX40), CD137 (4-1BB), FcεR1, NKG2D, CD244 (2B4), FcRγ, DAP10, DAP12, or CD3ζ.

23. The recombinant cell of claim 7, wherein the extracellular domain comprises a CD16A cleavage site.

24. The recombinant cell of claim 7, wherein the intracellular domain comprises a signaling domain.


SEQUENCE LISTING (33 sequences)

SEQ ID NO: 1 - protein, 335 aa, synthetic construct
MWFLTTLLLWVPVDGQVDTTKAVISLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGFFPPGYQVSFCLVMVLLFAVDTGLYFSVKTNIRSSTRDWKDHKFKWRKDPQDK

SEQ ID NO: 2 - protein, 374 aa, synthetic construct [sequence omitted]

SEQ ID NO: 3 - protein, 449 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDKRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - protein, 408 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - protein, 248 aa, Canis lupus
MWQLVSSTALLLLVSAGTQADVPKAVVVLEPKWNRVLTMDSVTLKCQGDHLLRDNYTWLHNGRPISNQISTYIIKNASIKNSGEYRCQTDQSKLSDPVQLEVHTGWLLLQVPRLVFQEGELIQLKCHSWKNTPVRNVQYFQNGRGKKFFYNNSEYHIPAATSEHNGSYFCRGIIGKKNESSEAVNIIIQGSSLPSTSLLLSHWPQIPFSLVMALLFAVDTGLYFAVQRDLRSSMGNLKNSKVIWSQGS

SEQ ID NO: 6 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 7 - protein, 372 aa, Canis lupus
MWLLTVLLLWVPAGAQTDPVKAVITLQPPWVSVFQEESVTLWCEGPHLPGDSSTQWFLNGTATQTLTPRYRIAAASVNDNGEYRCQTGLSVLSDPIQLGIHRDWLILQVSGRVFTEGEPLTLRCHGWNNKLVYNVLFYQNGTVLKFSPQNSEFTILKTTLHHNGIYHCSAMGKHRYESAGVSITIKELFPAPVLKASLSSPILEGHVVNLSCETKLLLQRPGLQLYFSFYMGSKTLLSRNTSSEYQILTAKKEDSGLYWCEATTEDGNVVKRSPELELQVVGPQTLTPVWFHVLFYVAMGMIFLVDTIFCMIIHKELQRKKKWNLEISLYSGLEKRVDSYLQKERDLEEPKYQELEQLQEKTPQKPPEGEQQ

SEQ ID NO: 8 - protein, 374 aa, Homo sapiens
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRKELKRKKKWDLEISLDSGHEKKVISSLQEDRHLEEELKCQEQKEEQLQEGVHRKEPQGAT

SEQ ID NO: 9 - protein, 374 aa, synthetic construct; identical to SEQ ID NO: 8

SEQ ID NO: 10 - protein, 374 aa, synthetic construct [sequence omitted]

SEQ ID NO: 11 - protein, 335 aa, synthetic construct
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGFFPPGYQVSFCLVMVLLFAVDTGLYFSVKTNIRSSTRDWKDHKFKWRKDPQDK

SEQ ID NO: 12 - protein, 530 aa, synthetic construct
MWFLTTLLLWVPVDGQVDTTKAVISLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGFFPPGYQVSFCLVMVLLFAVDTGLYFSVKTNIRSSTRDWKDHKFKWRKDPQDKRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - protein, 460 aa, synthetic construct
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGFFPPGYQVSFCLVMVLLFAVDTGLYFSVKTNIRSSTRDWKDHKFKWRKDPQDKRSKRSRLLHSDYMNMTPRRPGPTRKHYQPYAPPRDFAAYRSKRGRKKLLYIFKQPFMRPVQTTQEEDGCSCRFPEEEEGGCELRLKIQVRKAAITSYEKSDGVYTGLSTRNQETYETLKHEKPPQ

SEQ ID NO: 14 - protein, 440 aa, synthetic construct
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGFFPPGYQVSFCLVMVLLFAVDTGLYFSVKTNIRSSTRDWKDHKFKWRKDPQDKRSKRSRLLHSDYMNMTPRRPGPTRKHYQPYAPPRDFAAYRSKRGRKKLLYIFKQPFMRPVQTTQEEDGCSCRFPEEEEGGCELARPRRSPAQEDGKVYINMPGRG

SEQ ID NO: 15 - protein, 467 aa, synthetic construct
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGFFPPGYQVSFCLVMVLLFAVDTGLYFSVKTNIRSSTRDWKDHKFKWRKDPQDKRSKRSRLLHSDYMNMTPRRPGPTRKHYQPYAPPRDFAAYRSKRGRKKLLYIFKQPFMRPVQTTQEEDGCSCRFPEEEEGGCELGRLVPRGRGAAEAATRKQRITETESPYQELQGQRSDVYSDLNTQRPYYK

SEQ ID NO: 16 - protein, 510 aa, synthetic construct
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 343 aa, synthetic construct
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGAPREKYWLQFFIPLLVVILFAVDTGLFISTQQQVTFLLKIKRTRKGFRLLNPHPKPNPKNN

SEQ ID NO: 18 - protein, 335 aa, synthetic construct
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGFFPPGYQVSFCLVMVLLFAVDTGLYFSVKTNIRGAGRDWKDHKFKWRKDPQDK

SEQ ID NO: 19 - protein, 561 aa, synthetic construct
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGSNLFVASWIAVMIIFRIGMAVAIFCCFFFPSGGSGGGSGWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSGGSGGGSGRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 575 aa, synthetic construct
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGFFPPGYQVSFCLVMVLLFAVDTGLYFSVKTNIRSSTRDWKDHKFKWRKDPQDKWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSGGSGGGSGRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 505 aa, synthetic construct
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGFFPPGYQVSFCLVMVLLFAVDTGLYFSVKTNIRSSTRDWKDHKFKWRKDPQDKWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSGGSGGGSGRLKIQVRKAAITSYEKSDGVYTGLSTRNQETYETLKHEKPPQ

SEQ ID NO: 22 - protein, 485 aa, synthetic construct
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGFFPPGYQVSFCLVMVLLFAVDTGLYFSVKTNIRSSTRDWKDHKFKWRKDPQDKWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSGGSGGGSGARPRRSPAQEDGKVYINMPGRG

SEQ ID NO: 23 - protein, 512 aa, synthetic construct
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGFFPPGYQVSFCLVMVLLFAVDTGLYFSVKTNIRSSTRDWKDHKFKWRKDPQDKWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSGGSGGGSGGRLVPRGRGAAEAATRKQRITETESPYQELQGQRSDVYSDLNTQRPYYK

SEQ ID NO: 24 - protein, 615 aa, synthetic construct
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRKELKRKKKWDLEISLDSGHEKKVISSLQEDRHLEEELKCQEQKEEQLQEGVHRKEPQGATGWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSGGSGGGSGRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 248 aa, Canis lupus; identical to SEQ ID NO: 5

SEQ ID NO: 26 - protein, 372 aa, Canis lupus; identical to SEQ ID NO: 7

SEQ ID NO: 27 - protein, 287 aa, Canis lupus
MWLLTVLLLWVPAGAQTDWLILQVSGRVFTEGEPLTLRCHGWNNKLVYNVLFYQNGTVLKFSPQNSEFTILKTTLHHNGIYHCSAMGKHRYESAGVSITIKELFPAPVLKASLSSPILEGHVVNLSCETKLLLQRPGLQLYFSFYMGSKTLLSRNTSSEYQILTAKKEDSGLYWCEATTEDGNVVKRSPELELQVVGPQTLTPVWFHVLFYVAMGMIFLVDTIFCMIIHKELQRKKKWNLEISLYSGLEKRVDSYLQKERDLEEPKYQELEQLQEKTPQKPPEGEQQ

SEQ ID NO: 28 - DNA, 34 bp, synthetic construct
  source 1..34: mol_type = other DNA
cgggaattcggagacaacatgtggttcttgacaa

SEQ ID NO: 29 - DNA, 42 bp, synthetic construct
  source 1..42: mol_type = other DNA
ttggtacccaggtggaaagaagccaagcacttgaagctccaa

SEQ ID NO: 30 - DNA, 42 bp, synthetic construct
  source 1..42: mol_type = other DNA
ttggagcttcaagtgcttggcttctttccacctgggtaccaa

SEQ ID NO: 31 - DNA, 33 bp, synthetic construct
  source 1..33: mol_type = other DNA
ccggaattctcatttgtcttgagggtcctttct

SEQ ID NO: 32 - DNA, 34 bp, synthetic construct; identical to SEQ ID NO: 28
  source 1..34: mol_type = other DNA

SEQ ID NO: 33 - DNA, 33 bp, synthetic construct; identical to SEQ ID NO: 31
  source 1..33: mol_type = other DNA